(19) United States
(12) Patent Application Publication
(10) Pub. No.: US 2026/0165577 A1
(43) Pub. Date: Jun. 18, 2026

(54) VISUAL ACUITY USING A VISION SCREENING DEVICE

(71) Applicant: Welch Allyn, Inc., Skaneateles Falls, NY (US)

(72) Inventors: Matthew David Mullin, Memphis, NY (US); David L. Kellner, Baldwinsville, NY (US); Melissa R. Stancato, Syracuse, NY (US); Joshua F. Hess, Moravia, NY (US)

(21) Appl. No.: 19/339,208

(22) Filed: Sep. 24, 2025

Related U.S. Application Data
(60) Provisional application No. 63/733,304, filed on Dec. 12, 2024.

(51) Int. Cl. A61B 3/032; G06F 3/16

(52) U.S. Cl. CPC A61B 3/032; G06F 3/167

(57) ABSTRACT

A vision screening device for administering vision screening tests to a patient, and in particular vision screening test(s) to determine visual acuity results for eyes of a patient, is described herein. The vision screening device includes a sensor and a patient-facing display. The vision screening device is configured to receive input indicative of a selection of a predetermined distance for a visual acuity exam; determine, based on data from the sensor, a distance to a patient; determine an instruction for display at the second display to cause the distance to be within the threshold distance of the predetermined distance. The vision screening device is also configured to determine, based on the predetermined distance, a size for one or more digital objects of an optotype, and record responses to a visual acuity exam for determining a visual acuity response.

### CROSS-REFERENCE TO RELATED APPLICATIONS

This application claims priority to U.S. Provisional Application No. 63/733,304, titled “VISUAL ACUITY USING A VISION SCREENING DEVICE”, filed Dec. 12, 2024, which is hereby incorporated by reference in its entirety.

### TECHNICAL FIELD

This application is directed to medical equipment. In particular, this application is directed to a vision screening device, and associated systems and methods, for detection and assessment of diseases and disorders of the eye and performing visual acuity exams for a patient.

### BACKGROUND

Vision screening typically includes screening for diseases of the eye and visual acuity examinations. Such screening may include determining a visual acuity for each eye of a patient as well as screening using a plurality of both visible light, object recognition, color recognition, and infrared light tests to diagnosis a wide variety of potential eye diseases. For example, a transillumination test such as the Brückner red reflex test may be performed. During the red reflex test, the clinician illuminates the eye of the patient with visible light using an ophthalmoscope and examines the color and other characteristics of the light reflected back by the choroid and the retinal surfaces of the eye. Various diseases and abnormalities of the eyes can be detected using this test, such as corneal or media opacities, cataracts, and retinal abnormalities including tumors and retinoblastoma.

In addition, vision screening typically also includes one or more tests to determine various deficiencies associated with the patient's eyes. Such vision tests may include, for example, refractive error tests, accommodation tests, visual acuity tests, color vision screening and the like. One illustrative vision test involves the measurement of high contrast visual acuity. Visual acuity is a quantitative assessment of the ability to resolve high contrast optotypes. In the United States, the measurement is recorded in a ratio, such as 20/20, 20/40, 20/200, and so on. The ratio 20/20 indicates that at 20 feet, an individual is able to resolve a high contrast black letter which subtends 5 minutes of arc against a white background. From a test distance of 20 feet away, the 20/20 letter is 8.87 mm tall. The ratio 20/40 indicates that the individual can resolve a letter which is twice the size as the 20/20 benchmark. The ratio 20/200 means that the individual can resolve a letter that is ten times the size as the 20/20 benchmark.

Some of the vision screening tests require the use of infrared or near-infrared imaging, while other tests may require imaging under visible light, and/or a display screen to show content to the patient. However, ophthalmic testing devices such as a phoropter, autorefractor and photo-refractors, may only provide the capability to perform a limited range of tests. It would be advantageous to be able to screen for most vision problems and diseases using a single integrated device. Furthermore, due to the number of tests, it may be additional advantageous to be able to conduct a suite of test without repositioning the patient or recalibrating the machine. Current devices may require different distances to accurately conduct each test, resulting in movement of either the patient or the device between test and reducing the overall efficiency of the device.

The various examples of the present disclosure are directed toward overcoming one or more of the deficiencies noted above.

### SUMMARY

In an example of the present disclosure, a vision screening device includes a sensor such as a distance sensor or autofocus sensor, a first display disposed on a patient-facing side of the vision screening device, and a second display disposed on a user-facing side of the vision screening device opposite the patient-facing side. The device also includes one or more processors and one or more non-transitory computer-readable media having instructions stored thereon that, when executed by the one or more processors cause one or more processors to perform operations. The operations include receiving, via the first display, input indicative of a selection of a predetermined distance for a visual acuity exam, determining, based on data from the distance sensor, a distance to a patient, and determining, in response to the distance to the patient being outside a threshold distance of the predetermined distance, an instruction for display at the second display to cause the distance to be within the threshold distance of the predetermined distance. The operations also include determining, based on the predetermined distance, a size for one or more digital objects of an optotype and displaying, via the first display, the optotype.

In an example, the techniques described herein relate to a method for performing a visual acuity exam using a vision screening device including receiving, at a vision screening device, an input indicative of a selection of a predetermined distance for a visual acuity exam and determining, based on data from a sensor of the vision screening device such as an autofocus sensor or distance sensor, a distance to a patient. The method also includes determining, in response to the distance to the patient being outside a threshold distance of the predetermined distance, an instruction for display at a display of the vision screening device to cause the distance to be within the threshold distance of the predetermined distance, determining, based on the predetermined distance, a size for one or more digital objects of an optotype, and displaying, via a display of the vision screening device, the optotype.

In an example, the techniques described herein relate to a method including receiving, at a vision screening device, input data associated with initiating a visual acuity exam using the vision screening device, determining, using a sensor of the vision screening device, a distance from the vision screening device to a patient, and determining, based at least in part on the distance, a first optotype for display on a patient-facing display of the vision screening device. The method also includes receiving first response data at the vision screening device, the first response data indicative of a patient response to the first optotype, determining a second optotype for display on the patient-facing display of the vision screening device, receiving second response data at the vision screening device in response to the second optotype, and determining, using the vision screening device and in response to the first response data and the second response data, a visual acuity result for the patient.

### BRIEF DESCRIPTION OF THE DRAWINGS

Features of the present disclosure, its nature, and various advantages, may be more apparent upon consideration of the following detailed description, taken in conjunction with the accompanying drawings.

FIG. 1 illustrates a clinician using a vision screening device to perform one or more visual acuity exams for a patient, in accordance with one or more examples.

FIG. 2 illustrates an example vision screening device showing a clinician-facing display and interface, in accordance with one or more examples.

FIG. 3 illustrates a first perspective view of an example vision screening device, in accordance with one or more examples.

FIG. 4 illustrates a second perspective view of the example vision screening device of FIG. 3, in accordance with one or more examples.

FIG. 5 illustrates a perspective view of an example vision screening device with a patient facing cover removed, in accordance with one or more examples.

FIG. 6 illustrates a section view of an example vision screening device, in accordance with one or more examples.

FIG. 7 illustrates a section view of an example vision screening device, in accordance with one or more examples.

FIG. 8 illustrates a system architecture of a vision screening device, in accordance with one or more examples.

FIG. 9 illustrates a system architecture of a vision screening system for performing vision screening tests and recording test result data for patients, in accordance with one or more examples.

FIG. 10 illustrates a method for performing a visual acuity exam using the vision screening device, in accordance with one or more examples.

FIG. 11 illustrates a method for performing a visual acuity exam using the vision screening device for outputting an optotype as well as capturing and processing a response from a patient, in accordance with one or more examples.

FIG. 12 illustrates a method for performing a visual acuity exam for left and right eyes of a patient by adjusting an optotype presented between examination of the right and left eyes, in accordance with one or more examples.

FIG. 13 illustrates a method for recommending an optotype for use in a visual acuity exam using the vision screening device, in accordance with one or more examples.

### DETAILED DESCRIPTION

The present disclosure is directed to, in part, a vision screening device, and corresponding methods. Such an example vision screening device may be configured to perform one or more vision screening tests on a patient and to output the results of the vision screening test(s) to an operator of the device, such as a clinician or a physician's assistant. Specifically, the present disclosure is directed to devices and methods for performing visual acuity examinations for patients using a vision screening device that provides aids and guidance for clinicians using the vision screening device.

Visual acuity can be assessed at various distances. Among some of the reasons for measuring at particular distances include assessor preferences and space limitations within an office. The vision screening device described herein enables a clinician to perform a visual acuity assessment at a range of distances based on the space constraints and/or preferences of the individual. The vision screening device allows and accounts for the variation in distance while providing a consistent visual acuity result. Additionally, the vision screening device provides improvements for typical visual acuity workflows, for example by automatically presenting scrambled or rearranged orders of the digital optotypes to prevent patient memorization when moving from a first eye to a second eye.

The vision screening device provides a clinician with an option select a predetermined distance to the patient for performing the visual acuity test. In an example, the vision screening device gives the clinician the option to select a 10-foot visual acuity test or a 5-foot visual acuity test. The 5-foot distance may allow the clinician to perform all of the vision screening tests at a common distance, while the 10-foot distance may allow the clinician to perform a visual acuity test at a more traditional distance. Once the clinician selects the distance to use, the optotypes displayed on the vision screening device are adjusted to a proper size for that respective distance.

The vision screening device may also include a sensor that can be used to provide distance data for guiding the clinician to position the patient and/or vision screening device relative to one another such that the spacing meets the predetermined distance selected by the clinician. The sensor may be used by the vision screening device to determine a distance and guidance may be displayed on a display of the vision screening device instructing the clinician to move closer to the patient or further from the patient until within a threshold distance of the predetermined distance, at which point the vision screening device may begin the visual acuity exam. Furthermore, the vision screening device may conduct each of the screening tests from a common distance as to remove the requirement of moving the patient or recalibrating the machine.

The display on the vision screening device may not be large enough to display an entire eye chart for a visual acuity exam. Accordingly, the vision screening device may display only a portion of the eye chart such as a single line at a time or two lines at a time. To perform the visual acuity exam, the vision screening device may display multiple different optotypes representing different lines of an eye chart to the patient to determine the visual acuity result for the patient.

In an illustrative example, the vision screening device displays a critical line measurement (e.g., a line on a vision screening chart that represents a level of visual acuity that a person of a particular age group is expected to be able to read accurately). The clinician may score or record the response of the patient to the line. If the patient passes, the vision screening device automatically displays a next smaller line of characters. This process may then iteratively continue until the patient fails a line. If the patient fails the critical line, the vision screening device automatically displays a next larger line of characters. This process may also continue until the patient passes a line. In either scenario, the last line that a patient passes represents that patient's vision. The vision screening device may expedite the vision screening process for visual acuity exams by automatically displaying the next line for the patient based on their received response and therefore speeds up the workflow by reducing total number of button presses or interactions by a clinician.

In some examples the response from the patient may be recorded by the clinician marking on a display of the vision screening device whether the patient correctly or incorrectly replied to each character or object. For instance, objects that are correct may be tapped while incorrect objects may be unmarked or vice versa. At the completion of the line the vision screening device automatically proceeds to the next smaller or next larger line of the visual acuity chart based on the response of the patient.

In some examples the vision screening device may automatically record the response of the patient and score the response against the displayed optotype. For instance, the vision screening device may include a microphone used to receive audio data as the patient reads the displayed optotype and may process the audio data to determine the response (e.g., by using a transcription method or other such technique) and then comparing the received response against the optotype before proceeding to the next line of the visual acuity exam.

Based at least in part on the on analysis of the captured images by the vision screening device, the device may generate an output including at least one of a recommendation or a diagnosis associated with the patient. Such an output (e.g., the recommendation and/or the diagnosis) may be indicative of the visual acuity of one or both of the eyes of a patient. For example, the device may be used by a clinician to determine a visual acuity score for a left eye and for a right eye.

As the clinician moves from scoring the left eye to the right eye of the patient, the optotypes may be rearranged such that the characters initially presented in a first optotype at a first size for the right eye are presented in a second optotype at the first size for the left eye (or vice versa) but in a different order or arrangement. This may prevent a patient from memorizing the line during screening of a first eye and thereby lead to more accurate visual acuity results.

In addition to providing efficiencies for clinicians by automatically proceeding to a subsequent displayed optotype, the vision screening device may be used to determine and/or recommend an optotype or set of optotypes to present to the patient for the visual acuity exam. For instance, factors such as the patient age (e.g., and whether they are expected to be able to read letters), patient language, and/or geographic region may be used to recommend or determine a set of optotypes to present. The geographic information may be input by a clinician without requiring any additional patient information and may suggest a set of optotypes based on a language spoken by a majority of the population in the geographic region, or may provide the clinician with one or more options to select from based on the current region. After the geographic region is determined, the clinician may also input the patient age or other patient characteristic data, which may automatically be accessed from an electronic medical record if such data is available for access by the vision screening device, such as when in a clinician office storing or having access to such a patient record. The vision screening device may then recommend or determine an optotype to present based on the provided data. The vision screening device may present a recommendation for confirmation by the clinician or may automatically present the optotype, which may be changed or reconfigured by the clinician. The different optotypes may include different alphabet or language options as well as options for different optotypes such as Snellen charts, lea symbol charts, tumbling E charts, and other such optotypes.

In this manner, the vision screening device provides immediate access to a recommended optotype to use for each patient and that recommended optotype can then be displayed to the patient digitally. This allows the clinician to provide the best possible assessment for that particular patient, and speeds up workflow as the clinician does not have to sort through manual charts to find a proper one to display to the patient (if they even have the proper chart as some clinician resources or offices may be limited to only a small set of charts).

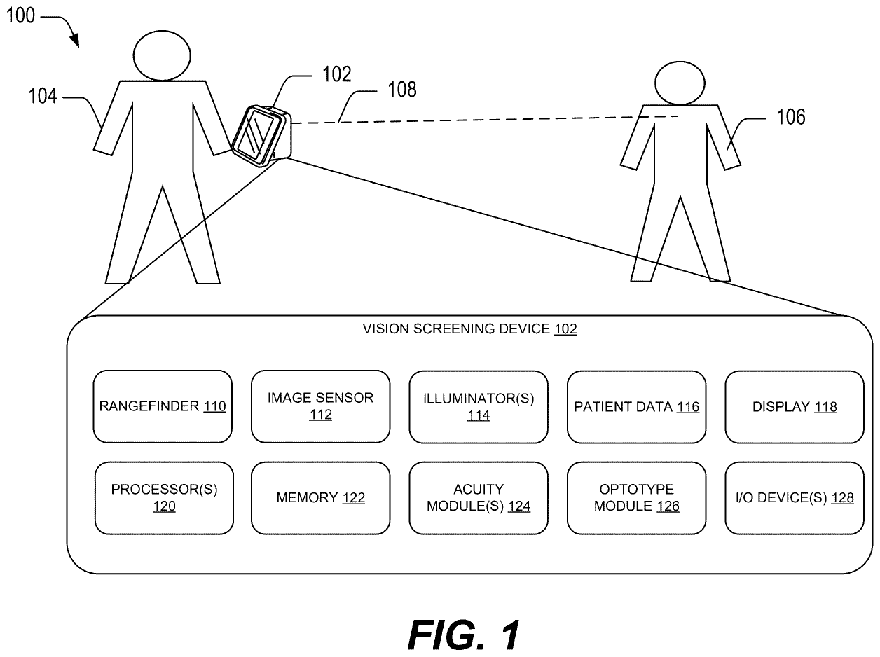

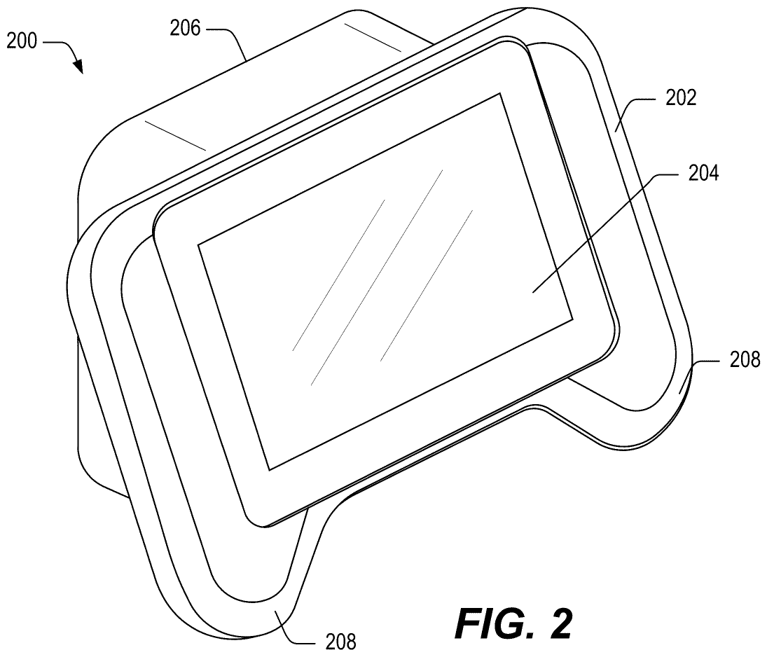

Turning now to the figures, FIG. 1 illustrates a system 100 with a clinician 104 using a vision screening device 102 to perform one or more visual acuity exams for a patient 106, in accordance with one or more examples. The clinician 104 may use the vision screening device 102, described in further detail with respect to FIGS. 2-9 below to perform the visual acuity exam among other exams for screening the patient 106 for one or more vision-based criteria. The vision screening device 102 may be used to perform the various exams on the patient 106 at a distance 108 from the patient. One or more different exams or screening processes may be performed on the patient 106. For example, a red reflex test, visual acuity exam, and other such tests may be performed on the patient 106 using the vision screening device 102. Visual acuity, and other exams may be assessed at various distances. Among some of the reasons for measuring at particular distances include assessor preferences and space limitations within an office. The vision screening device 102 described herein enables a clinician to perform a visual acuity exam and/or other exams at a range of distances based on the space constraints and/or preferences of the individual performing the exam. The vision screening device 102 allows and accounts for the variation in distance (e.g., to accurately provide optotypes of the correct size for a visual acuity exam) while providing a consistent visual acuity result. Additionally, the vision screening device 102 provides improvements for typical visual acuity workflows, for example by automatically presenting scrambled or rearranged orders of the digital optotypes to prevent patient memorization when moving from a first eye to a second eye.

The vision screening device 102 provides the clinician 104 with an option select a predetermined distance to the patient 106 for performing the visual acuity test. In an example, the vision screening device 102 gives the clinician 104 the option to select a 10-foot visual acuity test or a 5-foot visual acuity test. The 5-foot distance may allow the clinician 104 to perform all of the vision screening tests at a common distance, while the 10-foot distance may allow the clinician 104 to perform a visual acuity test at a more traditional distance. Once the clinician 104 selects the distance to use, the optotypes displayed on the vision screening device 102 are adjusted to a proper size for that respective distance.

The vision screening device 102 may be used for screening for diseases and abnormalities of the eyes of patients 106 as well as performing visual acuity exams for patients 106. In particular, the vision screening device 102 may include components for displaying optotypes, receiving inputs indicative of responses to the optotypes, generating recommendations for optotypes, capturing images of the eye(s) of the patient 106 under near-infrared as well as visible light radiation and conducting other ocular assessments such as mobility, color recognition, and/or visual acuity.

The vision screening device 102, shown and described in additional detail herein, at least with respect to FIGS. 2-9, includes a rangefinder 110, image sensor 112, illuminator(s) 114, storage for patient data 116, display 118, processor(s) 120, memory 122, acuity module(s) 124, optotype module 126, and I/O device(s) 128. The rangefinder 110 may include a sensor or other system for determining the distance 108 to the patient 106 from the vision screening device 102 such as an autofocus sensor or distance sensor. The rangefinder 110 provides distance data that may be used to determine the spacing between the vision screening device 102 and the patient 106. The rangefinder 110 may provide distance data such that the distance 108 may be determined to within a range of less than a foot (e.g., having a tolerance of up to one foot).

The vision screening device 102 includes components for controlling the emission of both near-infrared and visible light radiation (e.g., the illuminator(s) 114) and the corresponding components such as the image sensor 112 configured for capturing the reflected radiation from the eye(s) of the patient 106 during the screening. In examples, near-infrared images may be captured by the image sensor 112 before initiating the capture of visible light images, so that pupils of the eyes(s) of the patient 106 do not constrict, or accommodate, during the screening in response to visible light, and the screening may be completed without the need for dilation of the eyes. The vision screening device 102 may further include components for analyzing the captured images to determine and/or aid in diagnosing diseases, conditions, and/or abnormalities in the eye(s) of the patient 106, and components for determining and reporting of output(s) indicating the disease conditions and/or abnormalities detected during the screening.

The storage for patient data 116 may include storage devices for storing results of the tests or examinations performed using the vision screening device 102. In some examples such storage may be implemented as part of the memory 122 of the vision screening device 102 and/or may also include cloud-based or remote storage. In some examples the patient data 116 for the exams performed by the vision screening device 102 may be transmitted to a remote computing system and/or stored on-board the device for later use and consumption, such as by adding or inputting into an electronic medical record of the patient 106.

The display 118 includes a display that faces towards the patient 106 from the vision screening device 102 and provide optotypes or other displays for use in a vision screening exam, for example to display a portion of an eye chart for a visual acuity exam. The display 118 may include any suitable display of digital information that can be used to project information towards the patient 106.

The processor(s) 120 and memory 122 may be used to perform one or more operations as described herein. For example, the processor(s) 120 and memory 122 may be used to recommend optotypes for a visual acuity exam (e.g., through the optotype module 126) as well as to perform and/or aid in performance of the visual acuity exam (e.g., through the acuity module 124).

For example, the acuity module 124 may be used by the vision screening device 102 to provide the clinician 104 with an option select a distance 108 to the patient 106 for performing the visual acuity test. In an example, the vision screening device 102 gives the clinician 104 the option to select a 10-foot visual acuity test or a 5-foot visual acuity test. The 5-foot distance may allow the clinician 104 to perform all of the vision screening tests (e.g., including other tests such as a red reflex test or other such test that is performed at the 5-foot distance) at a common distance, while the 10-foot distance may allow the clinician 104 to perform a visual acuity test at a more traditional distance. Once the clinician 104 selects the distance to use, the optotypes displayed on the display 118 of the vision screening device 102 are adjusted to a proper size for that respective distance.

The vision screening device 102 may also use the rangefinder 110 to provide distance data and guidance for the clinician 104 to position the patient 106 and/or vision screening device 102 relative to one another such that the spacing meets the predetermined distance selected by the clinician 104. The rangefinder 110 and the distance data may be used to determine a distance and guidance may be displayed on a display of the vision screening device 102 instructing the clinician 104 to move closer to the patient 106 or further from the patient 106 until within a threshold distance of the predetermined distance, at which point the vision screening device 102 may begin the visual acuity exam using the acuity module 124. Furthermore, the vision screening device 102 may conduct each of the screening tests from a common distance as to remove the requirement of moving the patient 106 or recalibrating the vision screening device 102.

In some examples, the rangefinder 110 may include an autofocus system, the autofocus system may use sensors to detect a focal point and adjust the imaging components of the vision screening device 100 to increase or reach a maximum sharpness value. In some examples, the vision screening device 100 may identify areas of high contrast where distinct differences between adjacent pixels may be more obvious. The lens or other imaging components may adjust to change the plane of focus until the area of focus achieves maximum contrast. In some examples, the autofocus system may split incoming light into two separate images and send them to a dedicated autofocus sensor. The vision screening device 100 and specifically a computing component may compare the two images, and by comparing or measuring the displacement the vision screening device 100 may determine how far to move the lens or other imaging components to achieve focus. The autofocus system may also be used to determine or estimate the distance to the patient.

The display 118 on the vision screening device 102 may not be large enough to display an entire eye chart for a visual acuity exam. Accordingly, the vision screening device 102 may display only a portion of the eye chart such as a single line at a time or two lines at a time. To perform the visual acuity exam, the vision screening device 102 may display multiple different optotypes representing different lines of an eye chart to the patient to determine the visual acuity result for the patient 106.

In an illustrative example, the vision screening device 102 displays a critical line measurement (e.g., a line on a vision screening chart that represents a level of visual acuity that a person of a particular age group is expected to be able to read accurately). The clinician 104 may score or record the response of the patient 106 to the line. If the patient 106 passes, the vision screening device 102 automatically displays a next smaller line of characters. This process may then iteratively continue until the patient 106 fails a line. If the patient 106 fails the critical line, the vision screening device 102 automatically displays a next larger line of characters. This process may also continue until the patient 106 passes a line. In either scenario, the last line that a patient 106 passes represents that patient's vision. The vision screening device 102 may expedite the vision screening process for visual acuity exams by automatically displaying the next line for the patient based on their received response and therefore speeds up the workflow by reducing total number of button presses or interactions by a clinician.

In some examples the response from the patient 106 may be recorded by the clinician 104 marking on a display (e.g., the I/O device 128) of the vision screening device 102 whether the patient 106 correctly or incorrectly replied to each character or object. For instance, objects that are correct may be tapped while incorrect objects may be unmarked or vice versa. At the completion of the line the vision screening device 102 automatically proceeds to the next smaller or next larger line of the visual acuity chart based on the response of the patient 106.

In some examples the vision screening device 102 may automatically record the response of the patient 106 and score the response against the displayed optotype. For instance, the vision screening device 102 may include a microphone (e.g., as part of the I/O device 128) used to receive audio data as the patient 106 reads the displayed optotype and may process the audio data to determine the response (e.g., by using a transcription method or other such technique) and then comparing the received response against the optotype before proceeding to the next line of the visual acuity exam.

Based at least in part on the on analysis of the captured images by the vision screening device 102 and the received acuity data, the vision screening device 102 may generate an output including at least one of a recommendation or a diagnosis associated with the patient. Such an output (e.g., the recommendation and/or the diagnosis) may be indicative of the visual acuity of one or both of the eyes of a patient 106. For example, the device may be used by a clinician 104 to determine a visual acuity score for a left eye and for a right eye.

As the clinician moves from scoring the left eye to the right eye of the patient, the optotype module 126 may cause the optotypes to be rearranged such that the characters initially presented in a first optotype at a first size for the right eye are presented in a second optotype at the first size for the left eye (or vice versa) but in a different order or arrangement. This may prevent a patient from memorizing the line during screening of a first eye and thereby lead to more accurate visual acuity results.

In addition to providing efficiencies for clinicians by automatically proceeding to a subsequent displayed optotype, the vision screening device 102 may be used to determine and/or recommend an optotype or set of optotypes to present to the patient for the visual acuity exam through the optotype module 126. For instance, the optotype module 126 may receive factors such as the patient age (e.g., and whether they are expected to be able to read letters), patient language, and/or geographic region may be used to recommend or determine a set of optotypes to present to the clinician 104 for selection and use in the screening. The geographic information may be input by the clinician 104 without requiring any additional patient information and may suggest a set of optotypes based on a language spoken by a majority of the population in the geographic region, or may provide the clinician 104 with one or more options to select from based on the current region. After the geographic region is determined, the clinician 104 may also input the patient age or other patient characteristic data (which may automatically be accessed from an electronic medical record if such data is available for access by the vision screening device, such as when in a clinician office storing or having access to such a patient record. The optotype module 126 may then recommend or determine an optotype to present based on the provided data. The optotype module 126 may present a recommendation for confirmation by the clinician or may automatically present the optotype, which may be changed or reconfigured by the clinician. The different optotypes may include different alphabet or language options as well as options for different optotypes such as Snellen charts, lea symbol charts, tumbling E charts, and other such optotypes.

In this manner, the vision screening device provides immediate access to a recommended optotype to use for each patient and that recommended optotype can then be displayed to the patient digitally. This allows the clinician to provide the best possible assessment for that particular patient, and speeds up workflow as the clinician does not have to sort through manual charts to find a proper one to display to the patient (if they even have the proper chart as some clinician resources or offices may be limited to only a small set of charts).

Furthermore, the device may incorporate components, such as I/O device(s) 128, which may be configured to display images or graphics associated with visual acuity, mobility, and/or color recognition tests to the clinician 104 and also provides for the clinician 104 to interact with the vision screening device 102.

Additional details pertaining to the above-mentioned devices and techniques are described below with reference to the following figures. It is to be appreciated that while these figures describe devices and systems that may utilize the claimed methods, the methods, processes, functions, operations, and/or techniques described herein may apply equally to other devices, systems, and the like.

FIG. 2 illustrates a vision screening device 200 showing a clinician-facing display and interface, in accordance with one or more examples. The vision screening device 200 is illustrated with a housing 202 that encloses the components described below. The housing 202 is depicted with protrusions 208 that may enable a user to grasp the edges of the vision screening device 200 during use.

As illustrated in FIG. 2, in some examples an operator may administer vision screening tests, via a vision screening device 200, on a patient to determine eye health of the patient. As described herein, the vision screening device 200 may perform one or more vision screening tests, including screening for diseases and/or abnormalities of eye(s) when the eyes are illuminated by visible light. In addition, the vision screening device 200 may also be configured to perform other vision screening tests, such as a visual acuity test, a refractive error test, an accommodation test, dynamic eye tracking tests, color vision screening test and/or any other vision screening tests, configured to evaluate and/or diagnose the vision health of the patient. In examples, the vision screening device 200 may comprise a portable device configured to perform the one or more vision screening tests. Due to its portable nature, the vision screening device 200 may perform the vision screening tests at any location, from conventional screening environments, such as schools and medical clinics, to physician's offices, hospitals, eye care facilities, and/or other remote and/or mobile locations. Furthermore, the vision screening tests may be conducted at a common distance, providing for a more efficient process and resulting in the vision screening device 200 being capable of being used for large group testing. It is also envisioned that the vision screening device 200 may be used for administering vision screening tests to all age groups, including newborns and young children and geriatric patients.

As described herein, the vision screening device 200 may be configured to perform one or more vision screening tests on the patient. In examples, one or more vision screening tests may include illuminating the eye(s) of the patient with infrared or near-infrared (NIR) radiation and capturing reflected radiation from the eye(s) of the patient. For example, U.S. Pat. No. 9,237,846, the entire disclosure of which is incorporated herein by reference, describes systems and methods for determining refractive error based on photorefraction using pupil images captured under different illumination patterns generated by near-infrared (NIR) radiation sources. In other examples, vision screening tests, such as the red reflex test, may include illuminating the eye(s) of the patient with visible light, and capturing color image(s) of the eye(s) under visible light illumination. The vision screening device 200 may acquire data comprising color images and/or video data of the eye(s) under visible light illumination, and detect pupils, retinas, and/or lenses of the eye(s) of the patient. This data may be used to determine differences between left and right eyes, compare the captured images with standard images, or generate visualizations to assist the operator or a clinician in diagnosing diseases and abnormalities of the eye(s) of the patient. The vision screening device 200 may transmit the data to a vision screening system for analysis to determine an output associated with the patient. Alternatively, or in addition, the vision screening device 200 may perform some or all of the analysis locally to determine the output.

Indeed, in any of the examples described herein, some or all of the disclosed methods may be performed in whole or in part by the vision screening device 200 independently (e.g., without the vision screening system or its components), or by the vision screening system independently (e.g., without the vision screening device 200 or its components). For instance, in some examples, the vision screening device 200 may be configured to perform any of the vision screening tests, and/or other methods described herein without being connected to, or otherwise in communication with, the vision screening system. In some examples, the vision screening system may include one or more components that are similar to and/or the same as those included in the vision screening device 200, and thus, the vision screening system may be configured to perform any of the vision screening tests, and/or other methods described herein without being connected to, or otherwise in communication with, the vision screening device 200.

The vision screening device 200 may include one or more radiation source(s) (not shown in FIG. 2) configured to perform functions associated with administering one or more vision screening tests. The radiation source(s) may comprise individual radiation emitters, such as light-emitting diodes (LEDs), which may be arranged in a pattern to form an LED array. In examples, the radiation source(s) may include near-infrared (NIR) radiation emitters, such as NIR LEDs, for measuring the refractive error of the eye(s) of the patient using photorefraction methods. The NIR radiation emitters of the radiation source(s) may also be used for measuring the gaze angle or gaze direction of the eye(s) of the patient. In addition, the radiation source(s) may also include color LEDs for generating color stimuli for display to the patient during a color vision screening test.

The vision screening device 200 may also include one or more radiation sensor(s), such as infrared cameras, configured to capture reflected radiation from the eye(s) of the patient during the vision screening test(s). For example, the vision screening device 200 may emit, via the radiation source(s), one or more beams of radiation, and may be configured to direct such beams at the eye(s) of the patient. The vision screening device 200 may then capture, via the radiation sensor(s), corresponding radiation that is reflected back (e.g., from pupils of the eye(s)). In examples, the radiation sensor(s) may comprise NIR radiation sensor(s) to capture reflected NIR radiation while the eye(s) of the patient are illuminated by the NIR radiation source(s). The data captured by the NIR radiation sensor(s) may be used in the measurement of the refractive error and/or gaze angle(s) of the eye(s) of the patient. The data may include images and/or video of the pupils, retinas, and/or lenses of the eyes of the patient. In some examples, the images and/or video may be in grayscale (e.g., with values between 0 and 128, or between 0 and 256). The data may be captured intermittently, during specific periods of the vision screening test(s), or during the entire duration of the test(s). Additionally, the vision screening device 200 may process the image(s) and/or video data to determine change(s) in the refractive error and/or gaze angle(s) of the eye(s) of the patient. The grayscale images of the eye(s) captured under NIR illumination may also be used for screening for diseases and abnormalities of the eye(s) such as ametropia, strabismus, and occlusions.

In examples, the vision screening device 200 may further include visible white light source(s) and a camera configured to capture color images and/or video of the eyes under illumination by the white light source(s). The white light source(s) may comprise light-emitting diodes (LEDs) such as an array of LEDs configured to produce white light e.g., a blue LED with a phosphor coating to convert blue light to white light, or a combination of red, blue, and green LEDs configured to produce white light by varying intensities of individual red, blue and green LED activation. Individual LEDs of the array of LEDs may be arranged in a pattern configured to be individually operable to provide illumination from different angles during the vision screening test(s). The white light source(s) may also be configured to produce white light of different intensity levels. The camera may be configured to capture white light reflected from the eyes of the patient to produce digital color images and/or video. In some examples, pixel values in the color images and/or video may be in a RGB (red, green, blue) color space. The color images and/or video of the eye(s) captured under white light illumination may be used for screening for diseases and abnormalities of the eye(s) such as cataracts, media opacities in aqueous and vitreous humors, tumors, retinal cancers and detachment, and the like. In addition, the color images and/or video may be used in conjunction with the grayscale images captured under NIR illumination to generate visualizations to assist in the detection of a wide range of disease conditions of the eye(s).

The vision screening device 200 may also include one or more display screen(s), such as display 204, which may be color LCD (liquid crystal display), or OLED (organic light-emitting diode) display screens. The display 204 may be an operator display screen facing a direction towards the operator, configured to provide information related to the vision screening tests to the operator. In any of the examples described herein, the display 204 facing the operator may be configured to display and/or otherwise provide the output generated by the vision screening device 200 and/or generated by the vision screening system. The output may include testing parameters, current status and progress of the screening test(s), measurements(s) determined during the test(s), image(s) captured or generated during the screening test(s), a diagnosis determined based on one or more tests, and/or a recommendation associated with the diagnosis. The display 204 facing the operator may also display information related to or unique to the patient, and the patient's medical history.

In some examples, the vision screening device 200 may also include a display screen (not shown in FIG. 2) facing in a direction towards the patient and configured to display content to the patient. The content may include attention-attracting images and/or video to attract attention of the patient and hold the patient's gaze towards the vision screening device 200. Content corresponding to various vision screening test(s) may also be presented to the patient on the display screen. For example, the display screen may display color stimuli to the patient during a color vision screening test, or a Snellen eye chart during a visual acuity screening test. The display screens may be integrated with the vision screening device 200, or may be external to the device, and under computer program control of the vision screening device 200.

The vision screening device 200 includes additional sensors within the housing 202 including a sensor such as an autofocus sensor, range finder, an ambient light sensor, ambient infrared sensor, and other such sensors and components as described herein and as may be used by a clinician during a vision screening exam.

Depicted in FIG. 2, the vision screening device 200 includes a display 204 that may be used by a clinician or other user to interact with the vision screening device 200. The display 204 may be used for inputting information related to a patient, selecting test parameters, adding additional evaluation inputs, and otherwise controlling the vision screening device 200. As depicted, the display 204 may be a touch-screen or other similar display that enables user input through the display 204 while also providing output to the clinician.

On a side of the housing 202 opposite from the display 204 is a patient-facing surface 206. The patient-facing surface 206 includes an emission surface through which light and other radiation sources are emitted towards a patient during an exam. Additionally, the patient-facing surface 206 enables a sensor enclosed within the housing (not shown in FIG. 2) to gather distance data (e.g., from a distance sensor or autofocus sensor) for use in positioning the vision screening device 200 relative to a patient for evaluation.

The vision screening device 200 may be used for various evaluations at prescribed distances for distance vision testing. For example, vision testing and evaluation may be performed at a distance of five feet from the patient. The distance between the patient and the vision screening device is important for photorefraction exams (typically performed at a distance of three feet in previous systems) as well as visual acuity tests (typically tested at distances between ten and twenty feet). The vision screening device 200 is designed to perform the various tests and evaluation at a common distance for ease and speed of testing. The single common distance enables simpler exam processes as patients need not be shifted for different tests. Additional vision tests such as color vision and near vision may also be performed at the common distance (e.g., five feet or ten feet). Further still, to aid in vision screening, the vision screening device may be programmed to perform a sequence of exams simultaneously or back-to-back without requiring a reset or changing parameters of the vision screening device 200. In some examples, the clinician may use the vision screening device 200 to perform a suite of tests, or may select a subset of tests to perform during an evaluation.

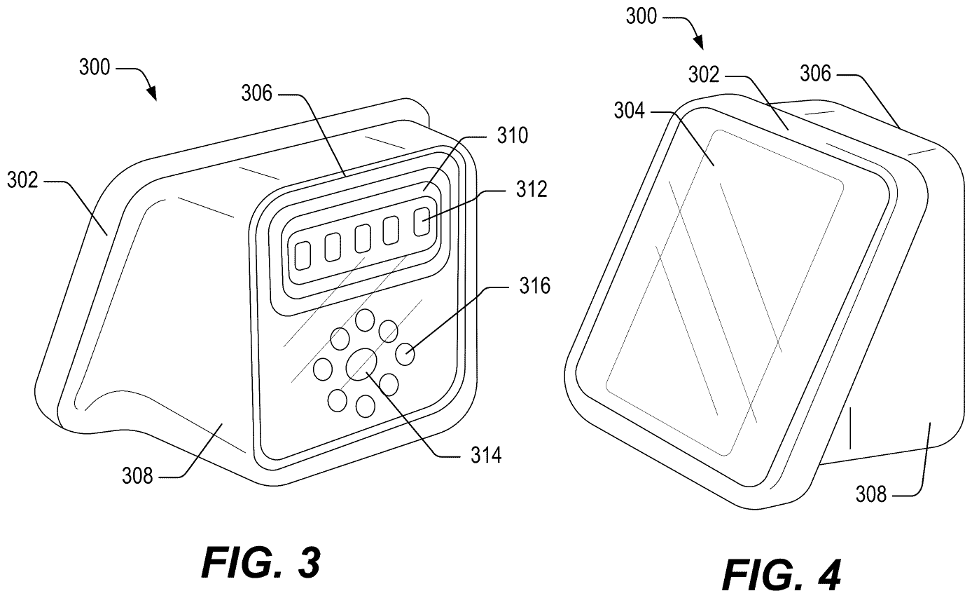

FIGS. 3-4 illustrate perspective views of an example vision screening device 300, in accordance with one or more examples. The example vision screening device 300 includes a housing 302 similar or identical to the housing 202. Additionally, the vision screening device 300 includes a display 304 similar or identical to the display 204 and an emission surface 306 on a patient-facing side. The emission surface 306 provides a surface through which light and/or other emissions may be projected towards a patient and also enables reflected light and other signals to be received. The housing 302 encloses a space 308 that receives reflected light and NIR light after it reflects off the patient. The emission surface 306 may be transparent to such light and signal transmissions, or include transparent portions, for example around a second display 310 and emitters 314 and aperture 316.

The second display 310 faces the patient and is used to display digital objects 312 such as letters, images, shapes, and other such digital display objects. The second display 310 may display the various digital objects 312 at different sizes or heights, for example to test visual acuity based on digital objects 312 of decreasing height. The second display 310 transmits digital information outwards towards the patient for the patient to interact with (e.g., read) while the emitters 314 and aperture 316 are used to gather information regarding the eyes of the patient.

The emitters 314 and aperture 316 are used to emit NIR light and white light towards the patient and receive reflected light off the patient at the aperture 316. The reflected light passes through the aperture 316 and into the interior of the housing 302 where it is reflected to a sensor (e.g., camera) or multiple cameras that may be used to detect the white light and NIR reflection data.

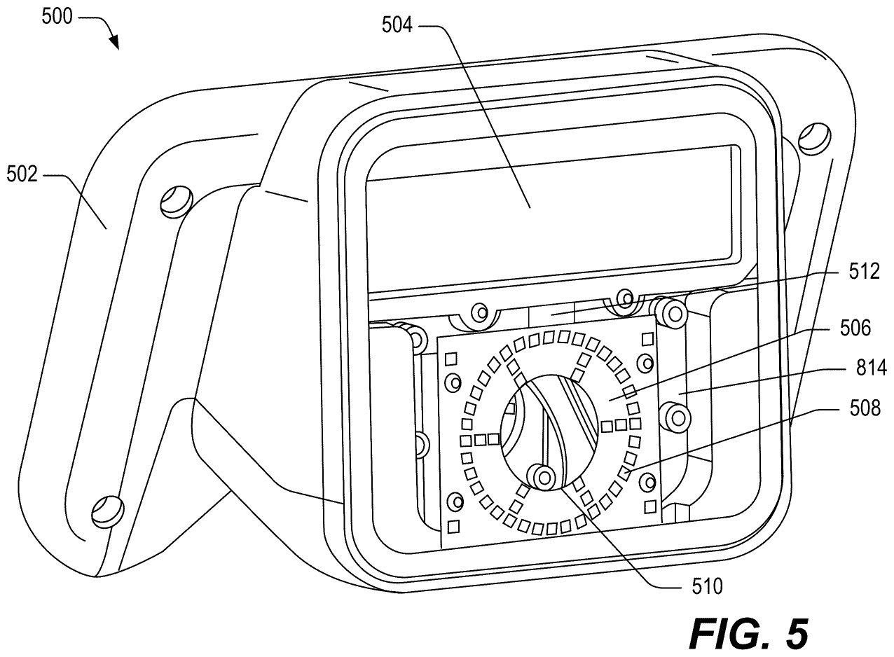

FIG. 5 illustrates a perspective view of an example vision screening device 500 with a patient facing cover removed, showing a partial view of internal components stored within a housing 502 of the vision screening device 500, in accordance with one or more examples. In addition to the housing 502, the vision screening device 500 includes a display 504 which serves as a patient-facing display to display digital objects such as symbols, pictures, letters, and other such information to a patient during an exam.

With the cover removed, an interior of the housing 502 is depicted including an emitting board 506 that includes emitters 508. The emitting board 506 provides power from a power source of the vision screening device 500 to the emitters 508 and also provides for a processor of the vision screening device 500 to control the emitters 508 to selectively emit radiation towards the patient. The emitters 508 may include NIR emitters as well as visible light emitters (e.g., white light emitters) that project light out of the patient-facing side of the vision screening device 500 towards the patient. The emitters 508 may include LEDs and other sources of near infrared light as well as visible light or other types of emitted radiation that may be directed towards the patient and received back at the vision screening device 500 after reflecting off the patient.

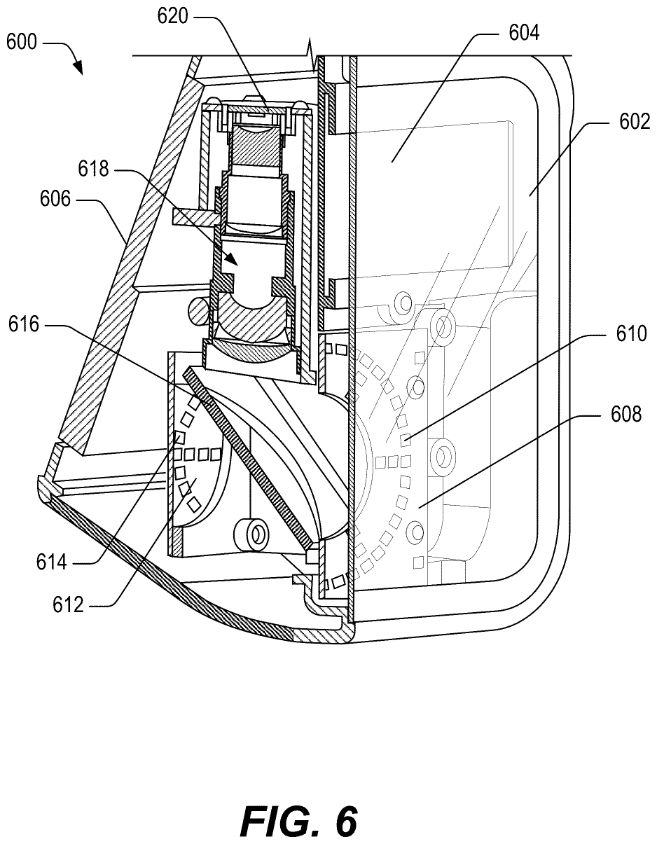

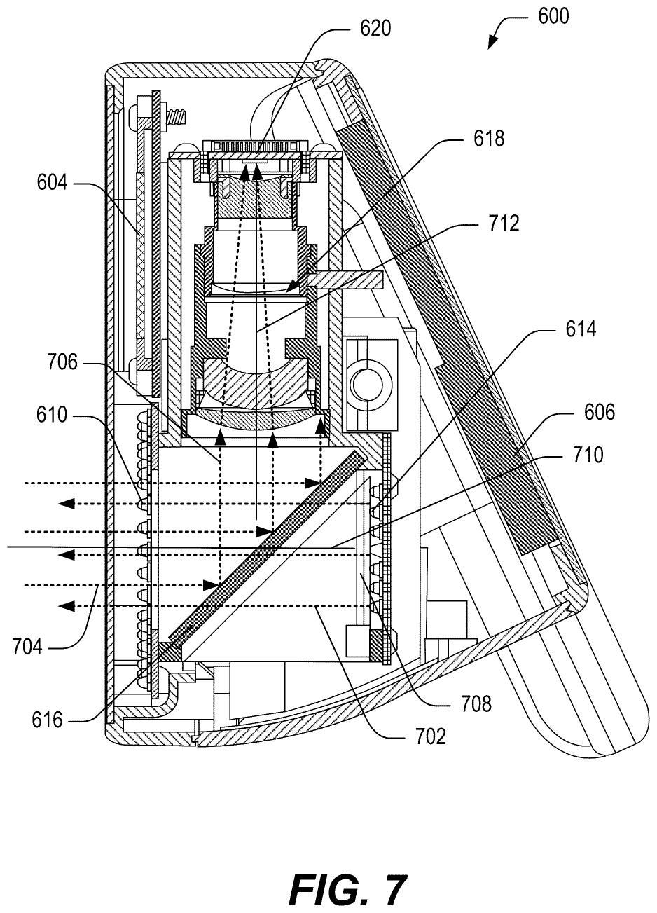

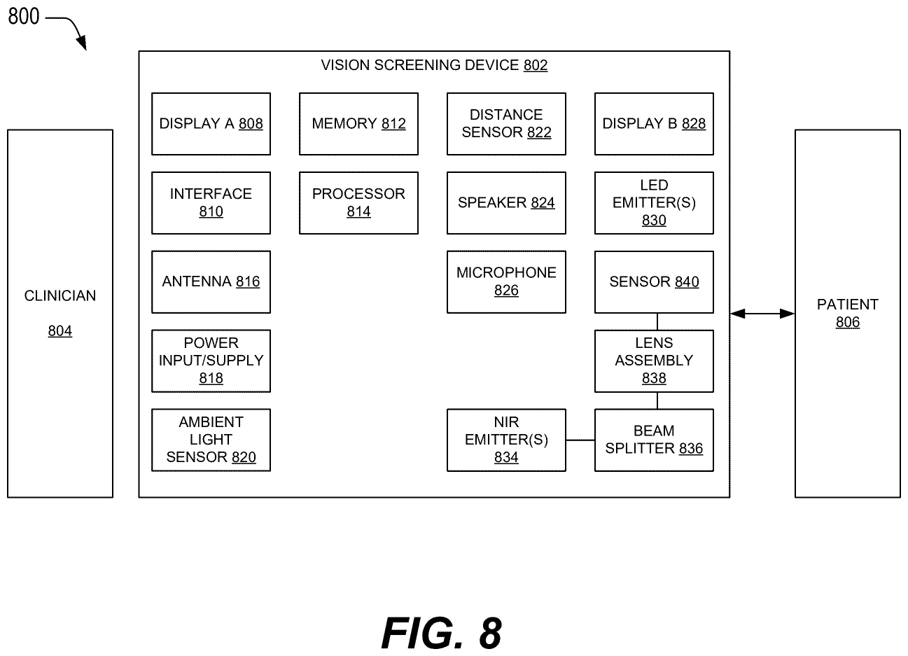

The emitting board 506 defines an opening 510 at a center of the array of the emitters 508. The array of emitters 508 may share an optical axis and may direct light or radiate energy outwards toward the patient along near parallel directions. The reflected energy from the patient passes through the opening 510 to reach further internal components of the vision screening device 500 for sensing and detection, as depicted in FIGS. 6-7.

The vision screening device 500 further includes a sensor 512. In FIG. 5 the sensor 512 is depicted positioned facing the patient and disposed vertically underneath the display 504. In some examples, the sensor 512 may be positioned within the housing 502 and any suitable position facing the patient-side of the housing 502 and oriented to face the patient when in use. The sensor 512 may include an ultrasonic sensor (and be positioned at the rear surface of the housing 502 with no glass between the distance sensor 512 and the patient, a millimeter wave radar sensor, a light-base sensor, or other such distance measuring sensor. In some examples, the sensor 512 may include an autofocus system, the autofocus system may use sensors to detect a focal point and adjust the imaging components of the vision screening device 500 to increase or reach a maximum sharpness value. In some examples, the vision screening device 500 may identify areas of high contrast where distinct differences between adjacent pixels may be more obvious. The lens or other imaging components may adjust to change the plane of focus until the area of focus achieves maximum contrast. In some examples, the autofocus system may split incoming light into two separate images and send them to a dedicated autofocus sensor. The vision screening device 500 and specifically a computing component may compare the two images, and by comparing or measuring the displacement the vision screening device 500 may determine how far to move the lens or other imaging components to achieve focus. The autofocus system may also be used to determine or estimate the distance to the patient.

The vision screening device 500 is shown with a rigid chassis 514 that floats within the housing 502. The optical components of the vision screening device including the emitters, beam splitters, lenses, and filters are aligned and calibrated and secured to the rigid chassis 514. In examples, the emitters of the LED boards, lenses, image sensors are all aligned and centered for capturing image data. The rigid chassis 514 floats within the housing, and may be mounted to the housing through one or more shock absorbers, energy absorbing devices, and other such components to provide protection for the components in the event the vision screening device 500 is dropped. In examples, one or more of the components, such as the clinician-facing display and/or patient-facing display may be surface mounted to a skin of the housing 502. The housing 502 further includes one or more access ports for servicing the interior components, for example to calibrate or re-align the optical components.

FIGS. 6-7 illustrate a section view of an example vision screening device 600, in accordance with one or more examples. In the section view, the vision screening device 600 is shown with elements of the vision screening device shown and described with respect to FIGS. 2-5 above, including a housing 602, display 604, display 606, emitting board 608, and emitters 610. The section view of FIG. 6 illustrates aspects of the interior of the vision screening device including a second emitting board 612, emitters 614, beam splitter 616, lens and filter assembly 618, and sensor 620.

Within the vision screening device 600, and positioned behind the emitting board 608 is a beam splitter 616 that is used to split light based on the direction from which the light is incident. Light reaching the beam splitter 616 from within the housing (e.g., behind the beam splitter 616) that is emitted by the emitters 614 of the second emitting board 612, is transmitted through a polarizer (not shown in FIG. 6) and then through the beam splitter and remains traveling in a direction parallel or substantially parallel with the direction or incident angle of the incoming light. The polarizer may be positioned between the beam splitter and the second emitting board 612. In contrast, reflected light that reaches the beam splitter 616 from the environment (and passes through an emitting surface of the vision screening device 600) is reflected by the beam splitter towards the lens and filter assembly 618 and finally to the sensor 620 where the reflected light is received and subsequently detected and processed as described herein. In some examples, the beam splitter 616 may be a plate beam splitter with a coating that causes the incident light originating from outside the vision screening device 600 to be reflected upwards (as oriented in FIG. 6) and into the lens and filter assembly 618. In examples, the beam splitter 616 may be accompanied with one or more additional filters or elements in the light path of the emitted NIR and/or visible light. The filter may, for example be a polarizing filter that polarizes the light as it is emitted from the vision screening device (e.g., after passing through the beam splitter from the emitters 614) and when the reflected light is returned, the beam splitter 616 may reflect the polarized light into the lens and filter assembly 618.

The lens and filter assembly 618 may include various filters including polarizing filters, filters to remove particular types of light, notch filters (e.g., to remove a portion of red light or other such light ranges), or otherwise treat the visible light and/or NIR light as it travels to the sensor 620. In examples, the filters may include coatings disposed on one or more lenses of the lens and filter assembly 618. The sensor 620 may include one or more sensors, such as a first sensor that detects NIR electromagnetic radiation and a second sensor that detects visible light. In examples, the sensor 620 may be a single sensor equipped to detect both NIR electromagnetic radiation as well as visible light. The light received at the sensor 620 may be processed based on the particular type of exam being performed either by an on-board processor and/or an external processing system to provide an output that may be displayed at the display 606 and/or output to an external system such as a system that hosts an electronic medical record for the patient.

FIG. 7 illustrates a section view of the vision screening device 600 and is illustrated including light traces of emitted radiation and received reflected radiation. Emitted light 702 includes light from emitters 614 that extends along a first optical axis 710 including along a direction parallel to the first optical axis 710 and may include visible night, NIR, white light, blue light, red light, green light, or other such light beams as emitted from the emitters 614. The emitted light 702 passes from the emitters 614 through a polarizer 708 and then through the beam splitter 616. The emitted light then passes out of the housing 602. Additional emitted light emanates from emitters 610 and joins the emitted light 702 traveling parallel with the first optical axis 710. After reflecting off an environment, including the patient, reflected light 704 is returned to the housing 602. The reflected light 704 may come in through an aperture in the housing 602 and may be parallel to the first optical axis but in a direction opposite the emitted light 702 then reaches the beam splitter 616 and some or all of the reflected light 704 is reflected to a second optical axis 712 perpendicular to the first optical axis 710 as diverted light 706. Examples, the diverted light 706 may include a portion of the reflected light 704. In examples the portion may be in a range of ten percent to one hundred percent. The second optical axis 712 enables the system to receive and process the diverted light 706 at the sensor 620 and provides for a greater distance over which the reflected light 704 and diverted light 706 may be conditioned through lenses and filters before reaching the sensor 620. The additional distance is enabled within a housing that maintains a compact handheld footprint that remains thing and similar in shape and form factor to a tablet.

FIG. 8 illustrates a system architecture 800 of a vision screening device 802, in accordance with one or more examples. The vision screening device 802 may be used by a clinician 804 interacting with a patient 806, as described herein. The system architecture 800 illustrates a subset of components that may be included as part of the vision screening device 802 in an illustrative example. The vision screening device 802 may include additional components not shown herein that may be used to perform the operations described herein and/or support one or more additional components. Further, other components may be substituted for one or more of the shown components that may perform the same or similar functions.

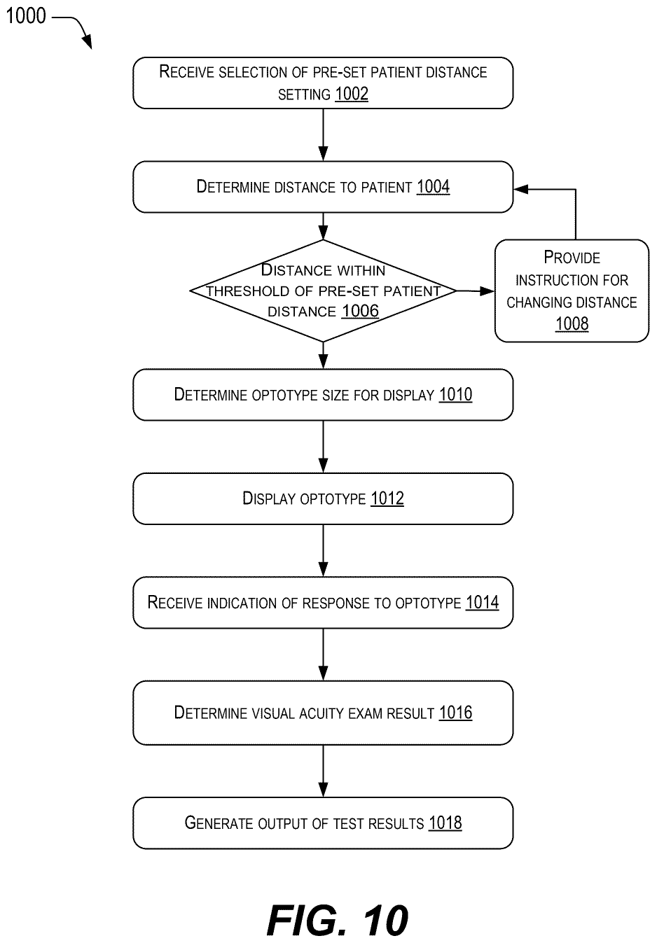

The vision screening device 802 includes a display A 808 that is clinician-facing and provides an interface for a clinician to input data as well as receive outputs from the vision screening device. A further interface 810, which may include a touchscreen display, provides the clinician with an ability to interact with the vision screening device, for example to input demographic data, patient data, select types of exams to perform, and other such interactions. A memory 812, such as a non-transitory computer-readable medium may include specific instructions stored thereon (e.g., software) that, when executed by a processor 814, cause the processor to perform various operations, such as the operations of FIGS. 10-14, or other operations described herein.

The vision screening device 802 further includes an antenna 816 that may provide a wireless communication with one or more other devices or systems such as a system that stores or provides access to electronic medical records for patients. A power input/supply 818 provides for portable power (e.g., batteries) as well as a system for charging batteries and/or providing consistent power to one or more other components of the vision screening device 802.

An ambient light sensor 820 may be used to detect light levels and/or characteristics of ambient light in the environment surrounding the visions screening device. The ambient light sensor 820 may detect a brightness of the environment and may also detect illumination colors, for instance if the lighting in a particular area is “warm” or “cool” relating to the temperature of the lighting. In some examples, the ambient light sensor 820 may be used to detect potential environmental light that may interfere with the vision screening tests, such as a prevalence of NIR light from a nearby source. Such information may be displayed on display A 808 for the clinician to either acknowledge or work to adjust environmental conditions to be more conducive to particular eye exams.

A sensor 822 may be used to determine a distance between the vision screening device 802 and the patient 806. The sensor 822 may include an autofocus sensor and/or autofocus system, a range finding sensor of any suitable type for detecting a distance between the location of the sensor (at the vision screening device 802) and the patient 806. In examples, the sensor 822 may be positioned behind a patient-facing cover on a side of the vision screening device 802 opposite display A 808. Accordingly, as with the imaging components, the sensor 822 may enable the housing of the vision screening device 802 to remain compact and user-friendly by presenting a slim and easily held device that is also portable.

A speaker 824 may provide audible outputs or cues, such as a beeping sound indicative of the patient being at or near the predetermined distance. In an example, an audible cue may increase in tone and/or frequency of a repeated sound as the patient 806 approaches the predetermined distance. A microphone 826 may be used to gather audible data, for example to enable the vision screening device 802 to recognize the patient reading a particular chart and to provide evaluation of their performance reading the symbols displayed on display B 828, that is user-facing, as described herein.

LED emitter(s) 830 may include NIR emitters 834 as well as white light emitters that provide light to reflect off the patient 806, with the reflected radiation received through a beam splitter 836 and lens assembly 838 before arriving at the sensor 840, as shown and described with respect to FIGS. 6-7 herein.

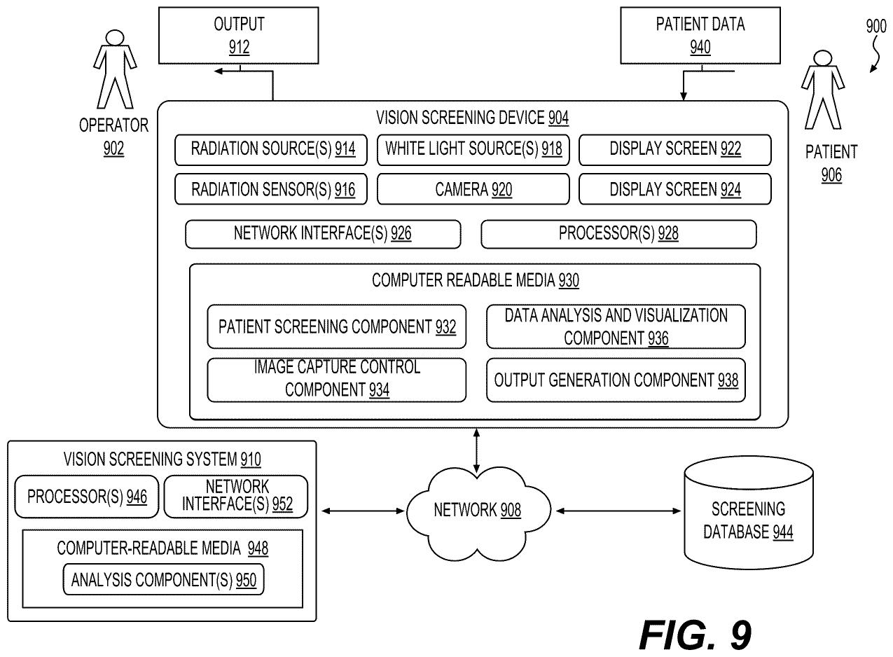

FIG. 9 illustrates a system architecture of a vision screening system for performing vision screening tests and recording test result data for patients, in accordance with one or more examples. As illustrated in FIG. 9, in some examples an operator 902 may administer vision screening tests, via a vision screening device 904, on a patient 906 to determine eye health of the patient 906. As described herein, the vision screening device 904 may perform one or more vision screening tests, including screening for diseases and/or abnormalities of eye(s) when the eyes are illuminated by visible light. In addition, the vision screening device 904 may also be configured to perform other vision screening tests, such as a visual acuity test, a refractive error test, an accommodation test, dynamic eye tracking tests, color vision screening test, visible light test, and/or any other vision screening tests, configured to evaluate and/or diagnose the vision health of the patient 906. In examples, the vision screening device 904 may comprise a portable device configured to perform the one or more vision screening tests. Due to its portable nature, the vision screening device 904 may perform the vision screening tests at any location, from conventional screening environments, such as schools and medical clinics, to physician's offices, hospitals, eye care facilities, and/or other remote and/or mobile locations. Furthermore, the vision screening tests may be conducted at a common distance, providing for a more efficient process and resulting in the vision screening device 904 being capable of being used for large group testing. It is also envisioned that the vision screening device 904 may be used for administering vision screening tests to all age groups, including newborns and young children and geriatric patients.

As described herein, the vision screening device 904 may be configured to perform one or more vision screening tests on the patient 906. In examples, one or more vision screening tests may include illuminating the eye(s) of the patient 906 with infrared or near-infrared (NIR) radiation and capturing reflected radiation from the eye(s) of the patient 906. For example, U.S. Pat. No. 9,237,846, the entire disclosure of which is incorporated herein by reference, describes systems and methods for determining refractive error based on photorefraction using pupil images captured under different illumination patterns generated by near-infrared (NIR) radiation sources. In other examples, vision screening tests, such as the red reflex test, may include illuminating the eye(s) of the patient 906 with visible light, and capturing color image(s) of the eye(s) under visible light illumination. The vision screening device 904 may acquire data comprising color images and/or video data of the eye(s) under visible light illumination, and detect pupils, retinas, and/or lenses of the eye(s) of the patient 906. This data may be used to determine differences between left and right eyes, compare the captured images with standard images, or generate visualizations to assist the operator 902 or a clinician in diagnosing diseases and abnormalities of the eye(s) of the patient. The vision screening device 904 may transmit the data, via a network 908, to a vision screening system 910 for analysis to determine an output 912 associated with the patient 906. Alternatively, or in addition, the vision screening device 904 may perform some or all of the analysis locally to determine the output 912. Indeed, in any of the examples described herein, some or all of the disclosed methods may be performed in whole or in part by the vision screening device 904 independently (e.g., without the vision screening system 910 or its components), or by the vision screening system 910 independently (e.g., without the vision screening device 904 or its components). For instance, in some examples, the vision screening device 904 may be configured to perform any of the vision screening tests, and/or other methods described herein without being connected to, or otherwise in communication with, the vision screening system 910 via the network 908. In other example, the vision screening system 910 may include one or more components that are similar to and/or the same as those included in the vision screening device 904, and thus, the vision screening system 910 may be configured to perform any of the vision screening tests, and/or other methods described herein without being connected to, or otherwise in communication with, the vision screening device 904.

As shown schematically in FIG. 9, the vision screening device 904 may include one or more radiation source(s) 914 configured to perform functions associated with administering one or more vision screening tests. The radiation source(s) 914 may comprise individual radiation emitters, such as light-emitting diodes (LEDs), which may be arranged in a pattern to form an LED array. In examples, the radiation source(s) 914 may include near-infrared (NIR) radiation emitters, such as NIR LEDs, for measuring the refractive error of the eye(s) of the patient 906 using photorefraction methods. The NIR radiation emitters of the radiation source(s) 914 may also be used for measuring the gaze angle or gaze direction of the eye(s) of the patient 906. In addition, the radiation source(s) 914 may also include color LEDs for generating color stimuli for display to the patient 906 during a color vision screening test.

The vision screening device 904 may also include one or more radiation sensor(s) 916, such as infrared cameras, configured to capture reflected radiation from the eye(s) of the patient during the vision screening test(s). For example, the vision screening device 904 may emit, via the radiation source(s) 914, one or more beams of radiation, and may be configured to direct such beams at the eye(s) of the patient 906. The vision screening device 904 may then capture, via the radiation sensor(s) 916, corresponding radiation that is reflected back (e.g., from pupils of the eye(s)). In examples, the radiation sensor(s) 916 may comprise NIR radiation sensor(s) to capture reflected NIR radiation while the eye(s) of the patient 906 are illuminated by the radiation source(s) 914. The data captured by the radiation sensor(s) 916 may be used in the measurement of the refractive error and/or gaze angle(s) of the eye(s) of the patient 906. The data may include images and/or video of the pupils, retinas, and/or lenses of the eyes of the patient 906. In some examples, the images and/or video may be in grayscale. The data may be captured intermittently, during specific periods of the vision screening test(s), or during the entire duration of the test(s). Additionally, the vision screening device 904 may process the image(s) and/or video data to determine change(s) in the refractive error and/or gaze angle(s) of the eye(s) of the patient 906. The grayscale images of the eye(s) captured under NIR illumination may also be used for screening for diseases and abnormalities of the eye(s) such as ametropia, strabismus, and occlusions. As described herein, in some examples, the radiation sensor(s) 916 may be combined with camera 920 into a single sensing component that detects both visible light and NIR.

In examples, the vision screening device 904 may further include visible white light source(s) 918 and camera 920 configured to capture color images and/or video of the eyes under illumination by the white light source(s) 918. The white light source(s) 918 may comprise light-emitting diodes (LEDs) such as an array of LEDs configured to produce white light e.g., a blue LED with a phosphor coating to convert blue light to white light, or a combination of red, blue, and green LEDs configured to produce white light by varying intensities of individual red, blue and green LED activation. Individual LEDs of the array of LEDs may be arranged in a pattern configured to be individually operable to provide illumination from different angles during the vision screening test(s). The white light source(s) 918 may also be configured to produce white light of different intensity levels. The camera 920 may be configured to capture white light reflected from the eyes of the patient to produce digital color images and/or video. The camera 920 may comprise a high-resolution, auto-focus digital camera with custom optics for imaging eyes in clinical applications. The color images and/or video captured by the camera 920 may be stored in various formats, such as JPEG, BITMAP, TIFF, etc. (for images) and MP4, MOV, WMV, AVI etc. (for video). In some examples, pixel values in the color images and/or video may be in a RGB (red, green, blue) color space. The color images and/or video of the eye(s) captured under white light illumination may be used for screening for diseases and abnormalities of the eye(s) such as cataracts, media opacities in aqueous and vitreous humors, tumors, retinal cancers and detachment, and the like. In addition, the color images and/or video may be used in conjunction with the grayscale images captured under NIR illumination to generate visualizations to assist in the detection of a wide range of disease conditions of the eye(s).

The vision screening device 904 may also include one or more display screen(s), such as display screen 922 and display screen 924, which may be color LCD (liquid crystal display), or OLED (organic light-emitting diode) display screens. The display screen 922 may be an operator display screen facing a direction towards the operator 902, configured to provide information related to the vision screening tests to the operator 902. In any of the examples described herein, the display screen 922 facing the operator 902 may be configured to display and/or otherwise provide the output 912 generated by the vision screening device 904 and/or generated by the vision screening system 910. The output 912 may include testing parameters, current status and progress of the screening test(s), measurements(s) determined during the test(s), image(s) captured or generated during the screening test(s), a diagnosis determined based on one or more tests, and/or a recommendation associated with the diagnosis. The display screen 922 facing the operator 902 may also display information related to or unique to the patient, and the patient's medical history.

In some examples, the vision screening device 904 may also include a display screen 924 facing in a direction towards the patient 906 and configured to display content to the patient 906. The content may include attention-attracting images and/or video to attract attention of the patient and hold the patient's gaze towards the vision screening device 904. Content corresponding to various vision screening test(s) may also be presented to the patient 906 on the display screen 924. For example, the display screen 924 may display color stimuli to the patient 906 during a color vision screening test, or a Snellen eye chart during a visual acuity screening test. The display screen 922 and display screen 924 may be integrated with the vision screening device 904, or may be external to the device, and under computer program control of the vision screening device 904.

The vision screening device 904 may transmit the data captured by the radiation sensor(s) 916 and/or the camera 920, via the network 908, using network interface(s) 926 of the vision screening device 904. In addition, the vision screening device 904 may also similarly transmit other testing data associated with the vision screening test(s) being administered, (e.g., type of test, duration of test, patient identification and the like). The network interface(s) 926 of the vision screening device 904 may be operably connected to one or more processor(s) 928 of the vision screening device 904, and may enable wired and/or wireless communications between the vision screening device 904 and one or more components of the vision screening system 910, as well as with one or more other remote systems and/or other networked devices. For instance, the network interface(s) 926 may include a personal area network component to enable communications over one or more short-range wireless communication channels, and/or a wide area network component to enable communication over a wide area network. In any of the examples described herein, the network interface(s) 926 may enable communication between, for example, the processor(s) 928 of the vision screening device 904, and the vision screening system 910, via the network 908. The network 908 shown in FIG. 9 may be any type of wireless network or other communication network known in the art. Examples of network 908 include the Internet, an intranet, a wide area network (WAN), a local area network (LAN), and a virtual private network (VPN), cellular network connections and connections made using protocols such as 802.11a, b, g, n and/or ac.

The vision screening system 910 may be configured to receive data, from the vision screening device 904 and via the network 908, collected during the administration of the vision screening test(s). In some examples, based at least in part on processing the data, the vision screening system 910 may determine the output 912 associated with the patient 906. For example, the output 912 may include a recommendation and/or diagnosis associated with eye health of the patient 906, based on an analysis of the color image data and/or NIR image data indicative of diseases and/or abnormalities associated with the eye(s) of the patient 906. The vision screening system 910 may communicate the output 912 to the processor(s) 928 of the vision screening device 904 via the network 908. As noted above, in any of the examples described herein one or more such recommendations, diagnoses, or other outputs may be generated, alternatively or additionally, by the vision screening device 904.

As described herein, a processor, such as the processor(s) 928, can be a single processing unit or a number of processing units, and can include single or multiple computing units or multiple processing cores. The processor(s) 928 can be implemented as one or more microprocessors, microcomputers, microcontrollers, digital signal processors, central processing units, state machines, logic circuitries, and/or any devices that manipulate signals based on operational instructions. For example, the processor(s) 928 can be one or more hardware processors and/or logic circuits of any suitable type specifically programmed or configured to execute the algorithms and processes described herein. As shown schematically in FIG. 9, the vision screening device 904 may also include computer-readable media 930 operably connected to the processor(s) 928. The processor(s) 928 can be configured to fetch and execute computer-readable instructions stored in the computer-readable media 930, which can program the processor(s) 928 to perform the functions described herein.

The computer-readable media 930 may include volatile and nonvolatile memory and/or removable and non-removable media implemented in any type of technology for storage of information, such as computer-readable instructions, data structures, program modules, or other data. Such computer-readable media 930 can include, but is not limited to, RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. The computer-readable media 930 can be a type of computer-readable storage media and/or can be a tangible non-transitory media to the extent that when mentioned, non-transitory computer-readable media exclude media such as energy, carrier signals, electromagnetic waves, and signals per se.

The computer-readable media 930 can be used to store any number of functional components that are executable by the processor(s) 928. In examples, these functional components comprise instructions or programs that are executable by the processor(s) 928 and that, when executed, specifically configure the one or more processor(s) 928 to perform actions associated with one or more of the vision screening tests used for the detection and diagnosis of diseases and abnormalities of the eye(s). For example, the computer-readable media 930 may store one or more functional components for administering vision screening tests, such as a patient screening component 932, an image capture control component 934, a data analysis and visualization component 936, and/or an output generation component 938, as illustrated in FIG. 9. At least some of the functional components of the vision screening device 904 will be described in detail below.

In examples, the patient screening component 932 may be configured to store and/or access patient data 940 associated with the patient 906. For example, the patient data 940 may include demographic information such as name, age, ethnicity, and the like. When the vision screening device 904 and/or vision screening system 910 initiates a vision screening test, the patient 906 may provide, or the operator 902 may request, from the patient 906 or a guardian of the patient 906 the patient data 940 regarding the patient's demographic information, medical information, preferences, and the like. In such examples, the operator 902 may request the data while the screening is in progress, or before the screening has begun. In some examples, the operator 902 may be provided with predetermined categories associated with the patient 906, such as predetermined age ranges (e.g., newborn to six months, six to twelve months, one to five years old, etc.), and may request the patient data 940 in order to select the appropriate category associated with the patient 906. In other examples, the operator 902 may be provided a free form input associated with the patient data 940. In still further examples, an input element may be provided to the patient 906 directly.

The vision screening device 904 may use a sensor such as an autofocus sensor or distance sensor to detect distance data corresponding to a distance between the vision screening device 904 and the patient 906. The distance may be used to position the vision screening device 904 relative to the patient, for example to reach a particular spacing or distance between the patient 906 and the vision screening device 904 such as described herein. Additional sensors such as ambient light sensors may be used for determining environmental conditions, such as whether environmental lighting conditions are suitable for a particular eye exam to take place.

Alternatively, or in addition, the vision screening device 904 and/or vision screening system 910 may determine and/or detect the patient data 940 during the vision screening test. For example, the vision screening device 904 may include one or more digital cameras, motion sensors, proximity sensors, or other image capture devices configured to collect images and/or video data of the patient 906, and one or more processors of the vision screening device 904 may analyze the data to determine the patient data 940, such as the age category of the patient 906 or a distance of the patient 906 from the screening device. For example, the vision screening device 904 may be equipped with a range finder, such as an ultra-sonic range finder, an infrared range finder, and/or any other proximity sensor that may be able to determine the distance of the patient 906 from the screening device.

Alternatively, or in addition, the vision screening device 904 may be configured to transmit the images/video data to the vision screening system 910, via the network 908, for analysis to determine the patient data 940. Further, the patient screening component 932 may be configured to receive, access, and/or store the patient data 940 associated with the patient 906 and/or additional patients. For example, the patient screening component 932 may store previous patient information associated with the patient 906 and/or other patients. For instance, the patient screening component 932 may store previous screening history of the patient 906, including data from previous screening such as color images, NIR images, and/or video of the eye(s) of the patient 906. The patient screening component 932 may receive the patient data 940 and/or may access such information via the network 908. For example, the patient screening component 932 may access an external database, such as screening database 944, storing data associated with the patient 906 and/or other patients. The screening database 84 may be configured to store the patient data 940 in association with a patient ID. When the operator 902 and/or the patient 906 enters the patient ID, the patient screening component 932 may access or receive the patient data 940 stored in association with the patient ID of the patient 906.

In examples, the patient screening component 932 may be configured to determine the vision screening test(s) to administer to the patient 906 based at least in part on the patient data 940. For example, the patient screening component 932 may utilize the patient data 940 to determine a testing category that the patient 906 belongs to (e.g., a testing category based on age, medical history, etc.). The patient screening component 932 may determine the vision screening test(s) to administer based on the testing category. For example, if the patient data 940 indicates that the patient is a newborn, the selected vision screening test(s) may include screening for congenital conditions of the eye such as congenital cataracts, retinoblastoma, opacities of the cornea, strabismus and the like. In addition, eye abnormalities may be associated with systemic inherited diseases such as Marfan syndrome and Tay-Sachs disease. For example, a screening test for a characteristic red spot in the eye may indicate Tay-Sachs disease. As another example, if the patient data 940 indicates that the patient is above fifty years old, the patient screening component 932 may determine that the vision screening test(s) include screening for onset of cataracts, macular degeneration and other age-related eye diseases.

The patient screening component 932 may also determine vision screening test(s) based on the patient's medical history. For example, the screening database 944 may store, in the patient data 940, medical history associated with previous vision screening tests of the patient 906, including test results, images of the eye(s), measurements, recommendations, and the like. The patient screening component 932 may access the patient data 940 including medical history from the screening database 944 and determine vision screening test(s) to administer to monitor status and changes in previously detected vision health issues. For example, if a progressive eye disease, such as onset of cataracts or macular degeneration, was detected in a previous screening, further screening may be administered to track the development of the disease. As another example, if the patient 906 had surgery for removal of a tumor of the eye(s), the vision screening test(s) may include screening for further tumors or scarring in the eye(s).

In some examples, the computer-readable media 930 may additionally store an image capture control component 934. The image capture control component 934 may be configured to operate the radiation source(s) 914, the radiation sensor(s) 916, the white light source(s) 918, and the camera 920 of the vision screening device 904, so that images of the eye(s) are captured under specific illumination conditions required for each particular vision screening test(s). As discussed, the radiation source(s) 914 may include NIR LEDs for illuminating the eye(s) during capture of grayscale images for measuring the refractive error and/or gaze angle of the eye(s) of the patient 906, and the white light source(s) 918 may include white light LEDs for illuminating the eye(s) during capture of color images of the eye(s) by the camera 920. In examples, the image capture control component 934 may generate commands to operate and control the individual radiation sources, such as LEDs of the NIR LEDs, as well as the LEDs of the white light source(s) 918. Control parameters of the LEDs may include intensity, duration, pattern and cycle time. For example, the commands may selectively activate and deactivate the individual LEDs of the radiation source(s) 914 and white light source(s) 918 to produce illumination from different angles as needed by the vision screening test(s) indicated by the patient screening component 932. The image capture control component 934 may activate the NIR LEDs of the radiation source(s) 914 used for measuring the refractive error and/or gaze angle of the eye(s) of the patient 906 in synchronization with the capture of images of the eye(s) by the radiation sensor(s) 916 during the performance of a vision screening test. Similarly, the image capture control component 934 may activate the LEDs of the white light source(s) 918 in synchronization with the capture of color images of the eye(s) by the camera 920.

The individual radiation sources, such as LEDs, of the radiation source(s) 914 or the white light source(s) 918 may be controlled by the image capture control component 934 according to control parameters stored in the computer-readable media 930. For instance, control parameters may include intensity, duration, pattern, cycle time, and so forth, of the NIR LEDs of the radiation source(s) 914 and/or the LEDs producing white light of the white light source(s) 918. For example, the image capture control component 934 may use the control parameters to determine a duration that individual LEDs of the radiation source(s) 914 and white light source(s) 918 emit radiation (e.g., 50 milliseconds, 800 milliseconds, 200 milliseconds, etc.). Additionally, the image capture control component 934 may utilize the control parameters to alter an intensity and display pattern of NIR LEDs of the radiation source(s) 914 for the determination of refractive error of the eye(s) based on photorefraction and/or gaze angle of the eye(s). With respect to intensity, the image capture control component 934 may control parameters to direct the LEDs of the white light source(s) 918 to emit light at an intensity that is bright enough to capture a color image of the eye(s) using the camera 920, while also limiting brightness to avoid or reduce pupil constriction or accommodation. The image capture control component 934 may also control the intensity of the white light source(s) 918 to gradually increase the intensity at a certain rate while activating the camera 920 to capture images and/or video of the eyes to record response of the pupils of the patient's eyes to the increasing intensity of illumination.

Further, the image capture control component 934 may order the emission of radiation from the radiation source(s) 914 and white light source(s) 918 so that the NIR LEDs are activated and the images of the eye(s) under NIR radiation are captured before the activation of the LEDs of the white light source(s) 918. In some examples, this ordering may prevent the constriction of the pupils of the eye(s) in response to white light impinging upon them, and/or may allow for the capture of images of the internal structures of the eye(s) without the need for dilating the pupils of the patient 906. In some examples, the image capture control component 934 may additionally control the radiation source(s) 914 and white light source(s) 918 to generate patterns such as circular patterns, alternating light patterns, flashing patterns, patterns of shapes such as circles or rectangles, and the like to attract the attention of the patient 906, and/or control color LEDs of the radiation source(s) 914 and white light source(s) 918 to display color stimuli such as color dot patterns to the patient 906 during vision screening.

The image capture control component 934 may also control the radiation sensor(s) 916 and the camera 920 to capture images and/or video of the eye(s) of the patient 906 during the administration of the vision screening test(s). For example, the radiation sensor(s) 916 may capture data indicative of reflected radiation from the eye(s) of the patient 906 during the activation of one or more of the radiation source(s) 914. The data may include grayscale image data and/or video data of the eye(s). The image capture control component 934 may synchronize the camera 920 to capture color image(s) and/or video data of the eye(s) with the activation of the white light source(s) 918 so that the eye(s) are illuminated by white light radiation during the capture of the color image and/or video data. In some examples, images of the left and the right eye may be captured under different illumination conditions (e.g., from a different individual source), so that the relative angle of illumination with the optical axis of the particular eye is the same for the left and the right eye. In other examples, images of both eyes may be captured simultaneously under the same illumination. As described herein, the image capture control component 934 of the vision screening device 904 may generate grayscale images of the eye(s) illuminated under NIR radiation, and color images of the eye(s) illuminated under white light. Capturing both the grayscale images and the color images may enable the detection of a wider range of diseases and abnormalities of the eyes.

In some examples, the computer-readable media 930 may also store a data analysis and visualization component 936. The data analysis and visualization component 936 may be configured to analyze the image and/or video data collected, detected, and/or otherwise captured by components of the vision screening device 904 (e.g., by the radiation sensor(s) 916, and the camera 920) during one or more vision screening tests. For example, the data analysis and visualization component 936 may analyze the data to determine location of the pupils of the eye(s) in the images, and identify a portion of the image(s) corresponding to the pupil (e.g., pupil image(s)). The data analysis and visualization component 936 may analyze the pupil image(s) to determine characterizations of appearance of the pupil(s) in the pupil image(s). For example, in the instance of the color image(s) captured by the camera 920, the characterizations may include an average color value, variance of color values, measure of uniformity, presence of inclusions, and the like. In the instance infrared image(s) captured by the radiation sensor(s) 916, the characterizations may include average grayscale value and variance of grayscale values, instead of the color, in addition to measures of uniformity and the presence of inclusions. The data analysis and visualization component 936 may further compare the left pupil image(s) and the right pupil image(s) to determine differences in appearance between the left and right pupils. For example, the differences may correspond to a difference in average color value or average grayscale value between the left pupil image(s) and right pupil image(s). The data analysis and visualization component 936 may also compare the pupil image(s) with standard pupil image(s) and/or pupil image(s) of the patient 906 captured during previous vision screening(s) to determine differences in appearance, such as differences in average color value or grayscale value, differences in the measure of uniformity, differences in detected inclusions, and the like. In any of the examples above, all captured image(s) or a subset of the captured grayscale and/or color images may be used to determine differences. In some examples, grayscale image(s) may not be used, and the difference may be determined based on the color image(s). It is to be noted that pixels of grayscale images may also be considered to have a color value, wherein the color value is determined by using the same grayscale value for each of the three-color channels (e.g., RGB). For example, a pixel with a grayscale value of 828, may be determined to have a color value of (128, 828, 828) in the RGB color space. The data analysis and visualization component 936 may also apply additional image processing steps to the grayscale image(s) and/or the color image(s) which may improve detection of disease states. For example, images may be sharpened, specific colors may be boosted or attenuated, color or brightness of the images may be balanced, and the like.

Further, the data analysis and visualization component 936 may be configured to receive, access, and/or analyze standard data associated with vision screening. For example, the data analysis and visualization component 936 may be configured to access or receive data from one or more additional databases (e.g., the screening database 944, a third-party database, etc.) storing testing data, measurements, and/or values indicating various thresholds or ranges within which measured values should lie. Such thresholds or ranges may be associated with patients having normal vision health and may be learned or otherwise determined from standard testing. The data analysis component and visualization component 936 may utilize the standard data for comparison with the average values and differences determined during the vision screening test(s) as described above. For example, the standard data may indicate a threshold or a range for a difference between color values of the left and right pupil images, where a difference greater than the threshold, or outside the range, corresponds to an abnormality in the eye(s) of the patient. Alternatively, or in addition, the data analysis and visualization component 936 may access a previous vision screening of the patient 906 and compare the values and differences with corresponding data from the previous screening(s). For example, an average color value of the pupil may be compared with an average color value from a previous screening to determine a difference. This difference may then be compared with standard thresholds or ranges to determine presence of an abnormality, as described above. Separate threshold(s) and/or range(s) may be indicated in the standard data for different types of diseases and abnormalities. In addition, the threshold(s) and/or range(s) associated with the vision screening test may also be based on the testing category of the patient 906 (e.g., the age group or medical history of the patient 906), where the threshold(s) and/or range(s) may be different for different testing categories. The data analysis and visualization component 936 may store as a part of the patient data 940, images and/or video captured or generated during the vision screening test(s), measurements associated with the vision screening test(s), test results, and other data in a database(e.g., in the screening database 944) for comparison of data over time to monitor vision health status and changes in vision health. In some examples, the stored images may include images of the face or partial face (e.g., eyes and part of nose) of the patient 906.

Based on the comparison with a threshold and/or range described above, the data analysis and visualization component 936 may generate a normal/abnormal or a pass/refer determination for each of the eyes of the patient 906. For example, if all values and differences measured are less than on equal to corresponding threshold(s), or fall within the corresponding range(s) of the standard data, a “normal” or “pass” determination may be made by the data analysis and visualization component 936, and an “abnormal” or “refer” determination made otherwise to indicate a referral for further screening. Alternatively, or in addition, the data analysis and visualization component 936 may generate a normal/abnormal determination for each of the diseases and/or abnormalities screened for during the vision screening session.

In examples, the data analysis and visualization component 936 may utilize one or more machine learning techniques to generate a diagnosis of specific diseases and/or types of abnormalities. For example, machine learning (ML) models may be trained with normal images of eyes, and images of eyes labeled as exhibiting various disease conditions and abnormalities. The trained ML model(s) may then generate an output indicating a disease or abnormality diagnosis when provided, as input, an image of the eye captured during the vision screening of the patient 906. In such examples, the data analysis and visualization component 936 may directly generate the output by providing an image of the eye as input to the trained ML model(s), without computing differences between pupil images or applying comparisons with a threshold and/or range. In some examples, a plurality of trained ML model(s) may be used, each ML model being trained to detect a specific disease or abnormality. In such examples, each ML model outputs a binary present/absent indication to indicate if the input image exhibits the disease or abnormality that the ML model is trained to detect. The data analysis and visualization component 936 may provide an image of the eye as input to each ML model of the plurality of trained ML model(s) for detecting one or more of a plurality of diseases and abnormalities. In examples, the ML models may be neural networks, including convolutional neural networks (CNNs). In other examples, the ML models can also include regression algorithms, decision tree algorithms, Bayesian classification algorithms, clustering algorithms, support vector machines (SVMs) and the like.

The data analysis and visualization component 936 may also generate visualizations of the eye(s) using the image(s) and/or video data captured by the radiation sensor(s) 916 and/or the camera 920. For example, a first visualization may include a composite image of the eye(s) incorporating both color information from the color image(s) and grayscale information from the grayscale image(s) captured by the radiation sensor(s) 916 under NIR illumination. The generation of the first visualization may include detection and identification of structures of the eye(s) such as pupils and/or lenses, followed by registration of the grayscale image(s) and the color image(s) so that the pupils are located in the same position in both types of image(s). The composite image may then be generated by using grayscale pixel values from the grayscale image(s) in some portions of the composite image and color pixel values from the color image(s) in other portions of the composite image. The portions of the composite image using grayscale pixel values and the portions using color pixel values may correspond to areas depicting different structures of the eye(s) (e.g., fovea, retina, cornea etc.). The composite image may more clearly delineate structures of the eye(s) for improved detection and assessment of diseases and/or abnormalities of the eye(s).

In another example, a second visualization may include a sequence of still images, or an animated video comprising the sequence of still images. In some instances, the sequence of images may be captured by the radiation sensor(s) 916 or the camera 920 while the eye(s) are illuminated by the radiation source(s) 914 or white light source(s) 918 at a progression of different angles along different axes with respect to the optical axis. In some examples, the visualization may include graphics and/or color-coding indicative of areas of the image of the eye(s) that are flagged as being abnormal. As described herein, the data analysis and visualization component 936 of the vision screening device 904 may process the grayscale images and the color images of the eye(s) captured during the administration of the vision screening test(s), to determine diseases and/or abnormalities associated with the eye(s) of the patient. In addition, the data analysis and visualization component 936 may generate, based on the grayscale and color images, visualizations of the eye(s) that aid a clinician or an operator of the vision screening device 904 to identify diseases and/or abnormalities of the eye(s).

The computer-readable media 930 may additionally store an output generation component 938. The output generation component 938 may be configured to receive, access, and/or analyze data from the data analysis and visualization component 936, and generate the output 912. For example, the output generation component 938 may utilize the normal/abnormal determinations of the data analysis and visualization component 936 to generate a recommendation in the output 912. The recommendation may indicate whether the screening results of the patient 906 indicate normal eye health, or further screening is needed based on one or more of the screening tests resulting in an “abnormal” finding. In addition, the output generation component 938 may incorporate all or a subset of the visualizations generated by the data analysis and visualization component 936 into the output 912 for aiding in diagnosis of the condition of the eye(s). Portions of the images and/or video captured by the radiation sensor(s) 916 or the camera 920 may also be included in the output 912. Additionally, if abnormality is determined, the output generation component 938 may incorporate a likely diagnosis into the output 912 based on the analysis by the data analysis and visualization component 936. The output 912 may be presented to the operator of the device via an interface of the device (e.g., on the display screen 922 of the vision screening device 904). In examples, the operator display screen may not be visible to the patient, e.g., the operator display screen may be facing in a direction opposite the patient. The output generation component 938 may also store the output 912, which may include a recommendation, diagnosis, measurements, captured images/video and/or the generated visualizations in a database, such as the screening database 944, for evaluation by a clinician, or for access during subsequent vision screening(s) of the patient 906. The screening database 944 may provide access to authorized medical professionals to enable printing of reports or further assessment of the data related to the screening of the patient 906.

Although FIG. 9 illustrates example processor(s) 928 and computer-readable media 930 storing a patient screening component 932, an image capture control component 934, a data analysis and visualization component 936, an output generation component 938 and/or other components and/or other items as components of the vision screening device 904, in any of the examples described herein, the vision screening system 910 may include similar components and/or the same components. In such examples, the vision screening system 910 may include processor(s) 946 and computer-readable memory 948 that are configured to perform the functions of some or all of the components in the computer-readable media 930 of the vision screening device 904. For example, one or more of the components of the computer-readable media 930 may be included in analysis component(s) 950 of computer-readable memory 948 and be executable by the processor(s) 946. In such examples, the vision screening system 910 may communicate with the vision screening device 904 using network interface(s) 952, and via the network 908, to receive data from the vision screening device 904 and send results (e.g., output 912), back to the vision screening device 904. The vision screening system 910 may be implemented on a computer proximate the vision screening device 904 or may be at a remote location. For example, the vision screening system 910 may be implemented as a cloud service on a remote cloud server.

The network interface(s) 952 may enable wired and/or wireless communications between the components and/or devices shown in system 900 and/or with one or more other remote systems, as well as other networked devices. For instance, at least some of the network interface(s) 952 may include a personal area network component to enable communications over one or more short-range wireless communication channels. Furthermore, at least some of the network interface(s) 952 may include a wide area network component to enable communication over a wide area network. Such network interface(s) 952 may enable, for example, communication between the vision screening system 910 and the vision screening device 904 and/or other components of the system 900, via the network 908. For instance, the network interface(s) 952 may be configured to connect to external databases (e.g., the screening database 944) to receive, access, and/or send screening data using wireless connections. Wireless connections can include cellular network connections and connections made using protocols such as 802.11a, b, g, and/or ac. In other examples, a wireless connection can be accomplished directly between the vision screening device 904 and an external system using one or more wireless protocols, such as Bluetooth, Wi-Fi Direct, radio-frequency identification (RFID), infrared signals, and/or Zigbee. Other configurations are possible. The communication of data to an external database can enable report printing or further assessment of the patient's visual test data. For example, data collected and corresponding test results may be wirelessly transmitted and stored in a remote database accessible by authorized medical professionals.

It should be understood that, while FIG. 9 depicts the system 900 as including a single vision screening system, in additional examples, the system 900 may include any number of local or remote vision screening systems substantially similar to the vision screening system 910 and configured to operate independently and/or in combination and configured to communicate via the network 908.

As discussed herein, FIG. 9 depicts a vision screening device 904 that includes components for administering vision screening tests to a patient. In some examples, one or more components may be implemented on a remote vision screening system communicating with the vision screening device 904 over a network 908.

FIGS. 10-14 illustrate methods for operating the vision screening device, in accordance with one or more examples. The example vision screening device may include one or more of the same components included in the vision screening devices as described herein. In some additional examples, the vision screening device can include different components that provide similar functions to the system 900 or other vision screening devices described herein.

FIGS. 10-14 provide flow diagrams illustrating example methods for vision screening, as described herein. The methods in FIGS. 10-14 are illustrated as collections of blocks in a logical flow graph, which represents sequences of operations that can be implemented in hardware, software, or a combination thereof. In the context of software, the blocks represent computer-executable instructions stored on one or more computer-readable storage media that, when executed by processor(s), perform the recited operations. Generally, computer-executable instructions include routines, programs, objects, components, data structures, and the like that perform particular functions or implement particular abstract data types. The order in which the operations are described is not intended to be construed as a limitation, and any number of the described blocks can be combined in any order and/or in parallel to implement the methods illustrated in FIGS. 10-14. In some embodiments, one or more blocks of the methods illustrated in FIGS. 10-14 can be omitted entirely. The operations described below with respect to the methods illustrated in FIGS. 10-14 can be performed by any of the devices or systems described above and/or by various components thereof.

FIG. 10 illustrates a method 1000 for performing a visual acuity exam using a vision screening device, according to at least one example. The method 1000 may be performed by the vision screening device as described herein. The vision screening device receives, at step 1002, a selection of a pre-set patient distance setting. The selection may be provided through an input device of the vision screening device, such as a touchscreen. The vision screening device then uses the distance sensor, autofocus system, or rangefinder of the vision screening device to determine a distance to the patient at step 1004. The distance to the patient may be determined based on data from the sensor and then be compared against the pre-set patient distance to determine if the distance to the patient is within a threshold distance of the pre-set patient distance at step 1006. The threshold may be a percentage of the distance to the patient and/or be based on the sensitivity or tolerance of the rangefinder. Accordingly, in some examples the threshold for a 10-foot setting may be greater than a threshold for a 5-foot setting for the distance.

In the event that the distance is not within the threshold distance of the pre-set patient distance at step 1006, then the method 1000 includes the vision screening device providing instruction for changing the distance at step 1008. The instruction may be presented on a clinician-facing display of the vision screening device and may show an indication of the actual distance determined from the distance sensor and the pre-set distance and may also include an indication of a direction of movement, for example directing the clinician to move towards or away from the patient. The process at step 1004, step 1006, and step 1008 may be repeated iteratively until the distance is within the threshold of the pre-set distance to the patient.

When the vision screening device determines that the distance to the patient is within the threshold of the pre-set patient distance, then the vision screening device determines an optotype size for display at step 1010. The vision screening device then determines a size for the optotype characters (or digital objects) based on the distance to the patient (e.g., the pre-set distance). The height of the digital objects is scaled according to the distance to the patient such that the visual acuity test may be performed. Accordingly, digital objects that should appear with a first height when viewed at a distance of 20 feet are scaled for 10-foot or 5-foot distances such that the visual acuity test may be performed consistently. The optotype may be scaled such that at a distance of 5 feet or 10 feet the digital objects appear to have the same height as they would appear to the patient from a distance of 20 feet during a typical visual acuity exam. The scaled optotype is then displayed at step 1012 for the patient to view.

At step 1014 the vision screening device receives an indication of the patient response to the optotype. The indication of the patient response may include a clinician input indicative of whether the patient responded accurately to each digital object of the displayed optotype. In some examples, the vision screening device may also receive audio data as the patient audibly responds to the displayed optotype and the vision screening device may process the audio data to transcribe the response and compare it against the displayed optotype. The results of the visual acuity exam may then be used to determine a visual acuity exam result at step 1016. The visual acuity exam may be performed at increasingly smaller digital object sizes on the displayed optotype until the patient responds incorrectly to the displayed digital objects. The final line that the patient is able to correctly read may then be used to determine the visual acuity of the patient and the output of the test result may be output at step 1018 through a display of the vision screening device and/or by outputting the result to an electronic medical record of the patient.

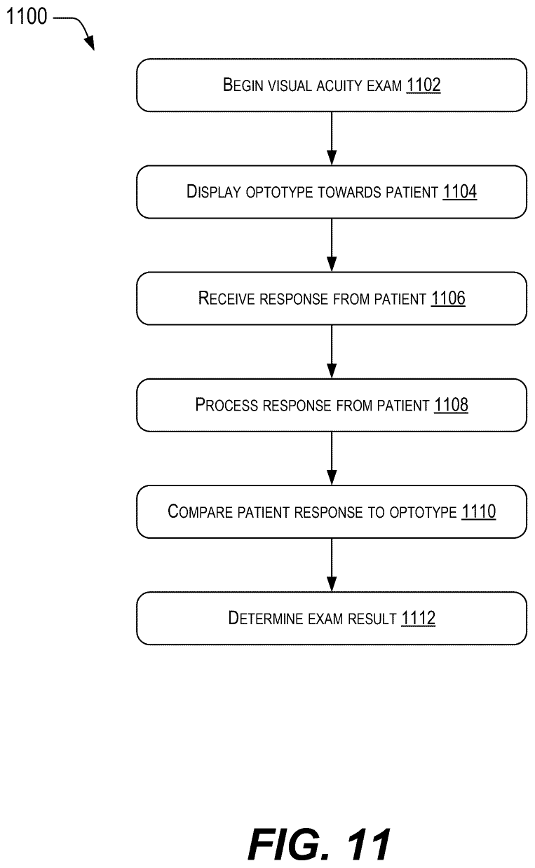

FIG. 11 illustrates a method 1100 for performing a visual acuity exam using the vision screening device for outputting an optotype as well as capturing and processing a response from a patient, in accordance with one or more examples. The method 1100 begins at step 1102 by initiating a visual acuity exam using the vision screening device, for example when a clinician selects that the visual acuity exam is to be performed on an input device of the vision screening device. The vision screening device displays an optotype towards the patient on the patient-facing display of the vision screening device at step 1104 and receives a response from the patient at step 1106. The response that is received may include an input response from a clinician or an audible response from the patient as they read or react to the displayed optotype. In an example, the clinician may mark, on the clinician-facing display, the correct and/or incorrect responses received from the patient. In an example, the vision screening device may use a microphone to capture audio data of the response to the optotype. In this example, the microphone may be engaged and/or audio data collected for a period of time following the display of the optotype towards the patient. The vision screening device may continue to capture audio data until it determines that the number of responses from the patient matches the number of digital objects included in the optotype.

The vision screening device processes the response from the patient at step 1108, for example to transcribe audio data into patient response data that may be compared against the optotype displayed at step 1110. The processed data may be compared for a match in the order as well as the identity of each of the digital objects. Based on the response and the comparison, the vision screening device determines the exam result at step 1112.

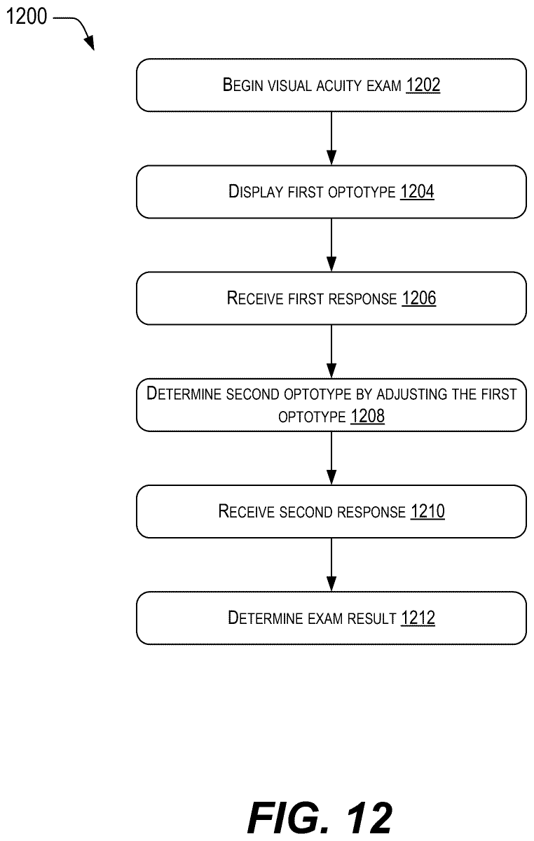

FIG. 12 illustrates a method 1200 for performing a visual acuity exam for left and right eyes of a patient by adjusting an optotype presented between examination of the right and left eyes, in accordance with one or more examples. The method 1200 begins at step 1202 by initiating a visual acuity exam using the vision screening device, for example when a clinician selects that the visual acuity exam is to be performed on an input device of the vision screening device. The vision screening device displays an optotype towards the patient on the patient-facing display of the vision screening device at step 1204 and receives a first response from the patient at step 1206. The response that is received may include an input response from a clinician or an audible response from the patient as they read or react to the displayed optotype. In an example, the clinician may mark, on the clinician-facing display, the correct and/or incorrect responses received from the patient. In an example, the vision screening device may use a microphone to capture audio data of the response to the optotype. In this example, the microphone may be engaged and/or audio data collected for a period of time following the display of the optotype towards the patient. The vision screening device may continue to capture audio data until it determines that the number of responses from the patient matches the number of digital objects included in the optotype.

After receiving the first response, the vision screening device determines a second optotype by adjusting or replacing the first optotype at step 1208. The second optotype may include a line of an eye exam below or above the line presented in the first optotype. For example, the first optotype may have digital objects of a first size and the second optotype may have digital objects of a second size. The second size may be less than the first size in the event that the first response corresponds or matches the first optotype, indicating that the patient successfully cleared the first optotype. The second size may be greater than the first size in the event that the first response does not correspond to or match the first optotype, for example if the patient responds incorrectly to one or more of the digital objects of the first optotype. In some examples, the second optotype is automatically displayed in response to the first response matching or corresponding to the first optotype without requiring any input from the clinician.

Following the display of the second optotype, the vision screening device receives a second response at step 1210 in a manner similar or identical to the way the first response is received. The method 1200 may repeat the iterative displaying of optotypes of increasing or decreasing size until the patient incorrectly responds to one of the displayed optotypes. The vision screening device then outputs an exam result at step 1212 on the clinician-facing display of the vision screening device.

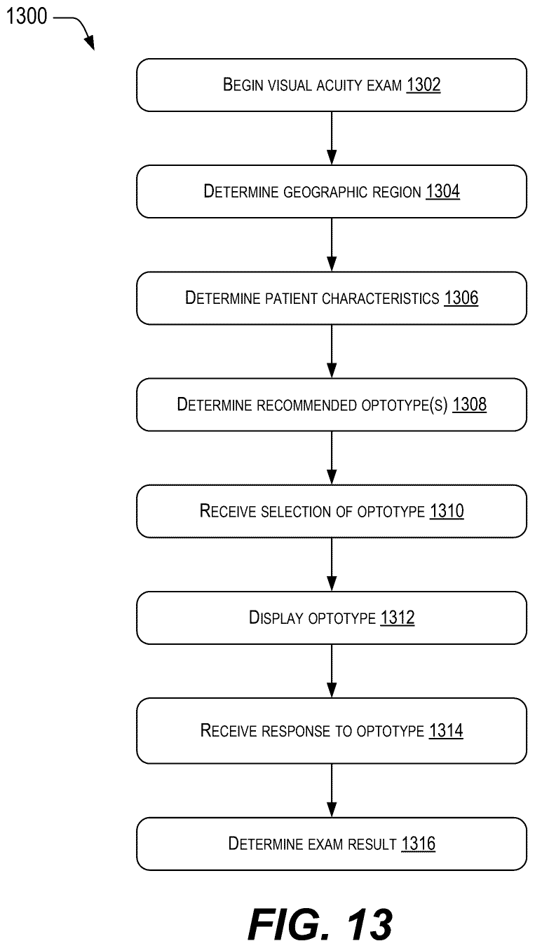

FIG. 13 illustrates a method 1300 for recommending an optotype for use in a visual acuity exam using the vision screening device, in accordance with one or more examples. The method 1300 begins at step 1302 by initiating a visual acuity exam using the vision screening device, for example when a clinician selects that the visual acuity exam is to be performed on an input device of the vision screening device. At step 1304, the method includes determining a geographic region for the screening to take place. In some examples the geographic region may be input by a clinician and/or pre-set based on factory settings and/or owner settings. The geographic region may also be determined based on a geolocator and/or location determining system of the device that uses internet-based location services or other such locating systems to provide an approximate location for the geographic region.

At step 1306, the vision screening device determines patient characteristics for the patient to be screened. The patient characteristics may include the age, language preferences, or other such information for the patient. The patient characteristics may be input by the clinician and/or accessed from a patient medical record of a system in communication with the vision screening device.

The vision screening device determines one or more recommended optotypes in response to the geographic region and/or the patient characteristics at step 1308. The vision screening device may determine and/or recommend an optotype or set of optotypes to present to the patient for the visual acuity exam. For instance, factors such as the patient age (e.g., and whether they are expected to be able to read letters), patient language, and/or geographic region may be used to recommend or determine a set of optotypes to present. The geographic information may suggest a set of optotypes based on a language spoken by a majority of the population in the geographic region, or may provide the clinician with one or more options to select from based on the current region. The patient characteristic data may automatically be accessed from an electronic medical record if such data is available for access by the vision screening device, such as when in a clinician office storing or having access to such a patient record. The vision screening device may recommend or determine an optotype to present based on the provided data. The vision screening device may present a recommendation for confirmation by the clinician or may automatically present the optotype, which may be changed or reconfigured by the clinician. The different optotypes may include different alphabet or language options as well as options for different optotypes such as Snellen charts, lea symbol charts, tumbling E charts, and other such optotypes. In this manner, the vision screening device provides immediate access to a recommended optotype to use for each patient and that recommended optotype can then be displayed to the patient digitally. This allows the clinician to provide the best possible assessment for that particular patient, and speeds up workflow as the clinician does not have to sort through manual charts to find a proper one to display to the patient (if they even have the proper chart as some clinician resources or offices may be limited to only a small set of charts).

At step 1310, the clinician may select a recommended optotype or confirm the recommended optotype. In some examples the vision screening device may default to a first recommended optotype for display unless overridden by a clinician-selection. The selected optotype is displayed at step 1312 and a response received at step 1314, similar to in the other methods described herein, before determining and/or outputting exam results at step 1316.

The foregoing is merely illustrative of the principles of this disclosure and various modifications can be made by those skilled in the art without departing from the scope of this disclosure. The above-described examples are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

As a further example, variations of apparatus or process limitations (e.g., dimensions, configurations, components, process step order, etc.) can be made to further optimize the provided structures, devices and methods, as shown and described herein. In any event, the structures and devices, as well as the associated methods, described herein have many applications. Therefore, the disclosed subject matter should not be limited to any single example described herein, but rather should be construed in breadth and scope in accordance with the appended claims.

What is claimed is:

1. A vision screening device, comprising:
a sensor system for determining distance data;
a first display disposed on a patient-facing side of the vision screening device;
a second display disposed on a user-facing side of the vision screening device opposite the patient-facing side;
one or more processors; and
one or more non-transitory computer-readable media having instructions stored thereon that, when executed by the one or more processors cause one or more processors to:
receive, via the first display, input indicative of a selection of a predetermined distance for a visual acuity exam;
determine, based on data from the sensor, a distance to a patient;
determine, in response to the distance to the patient being outside a threshold distance of the predetermined distance, an instruction for display at the second display to cause the distance to be within the threshold distance of the predetermined distance;
determine, based on the predetermined distance, a size for one or more digital objects of an optotype; and
display, via the first display, the optotype.

2. The vision screening device of claim 1, wherein the predetermined distance comprises at least one of five feet, ten feet, or twenty feet.

3. The vision screening device of claim 1, wherein the instructions comprise further instructions to cause the one or more processors to additionally:
receive result data indicative of a patient response to the optotype; and
determine, based on the result data, a visual acuity result for the patient.

4. The vision screening device of claim 3, wherein the result data is received through the second display.

5. The vision screening device of claim 3, further comprising a microphone, and wherein the result data is associated with audio data received through the microphone.

6. The vision screening device of claim 5, wherein the audio data is processed using a language recognition module to generate the result data, and the visual acuity result is based on a comparison of the result data against the optotype.

7. The vision screening device of claim 3, wherein the instructions comprise further instructions to cause the one or more processors to additionally:
in response to the result data, display a second optotype at the first display, wherein:
the second optotype comprises a second digital object having a second size less than the size in response to the result data corresponding with the optotype; and
the second optotype comprises a third digital object having a third size greater than the size in response to the result data differing from the optotype by at least a threshold.

8. A method comprising:
receiving, at a vision screening device, an input indicative of a selection of a predetermined distance for a visual acuity exam;
determining, based on data from a sensor of the vision screening device, a distance to a patient;
determining, in response to the distance to the patient being outside a threshold distance of the predetermined distance, an instruction for display at a display of the vision screening device to cause the distance to be within the threshold distance of the predetermined distance;
determining, based on the predetermined distance, a size for one or more digital objects of an optotype; and
displaying, via a display of the vision screening device, the optotype.

9. The method of claim 8, further comprising:
receiving input data associated with a patient response to the optotype; and
determining a visual acuity result based on a comparison of the input data and the optotype.

10. The method of claim 9, wherein receiving the input data comprises:
receiving audio data through a microphone of the vision screening device; and
determining the input data by processing the audio data.

11. The method of claim 8, further comprising:
determining a geographic region for the visual acuity exam;
determining one or more patient characteristics; and
determining a recommended optotype, wherein the optotype is based on the recommended optotype.

12. The method of claim 11, further comprising determining the optotype based on the recommended optotype and a user input of the vision screening device.

13. The method of claim 8, further comprising:
receiving result data in response to the optotype; and
in response to the result data, display a second optotype at the display, wherein:
the second optotype comprises a second digital object having a second size less than the size in response to the result data corresponding with the optotype; and
the second optotype comprises a third digital object having a third size greater than the size in response to the result data differing from the optotype by at least a threshold.

14. The method of claim 8, wherein the predetermined distance comprises at least one of five feet, ten feet, or twenty feet.

15. A method comprising:
receiving, at a vision screening device, input data associated with initiating a visual acuity exam using the vision screening device;
determining, using a sensor of the vision screening device, a distance from the vision screening device to a patient;
determining, based at least in part on the distance, a first optotype for display on a patient-facing display of the vision screening device;
receiving first response data at the vision screening device, the first response data indicative of a patient response to the first optotype;
determining a second optotype for display on the patient-facing display of the vision screening device;
receiving second response data at the vision screening device in response to the second optotype; and
determining, using the vision screening device and in response to the first response data and the second response data, a visual acuity result for the patient.

16. The method of claim 15, wherein the first optotype comprises a first arrangement of digital objects and the second optotype comprises a second arrangement of the digital objects, the second arrangement different from the first arrangement.

17. The method of claim 15, wherein the first optotype is for a first eye of the patient and the second optotype is for a second eye of the patient.

18. The method of claim 15, wherein:
determining the first optotype for the display comprises scaling a set of digital objects to a first size based on the distance and arranged in a first arrangement; and
determining the second optotype for the display comprises the set of digital objects at the first size in a second arrangement different from the first arrangement.

19. The method of claim 18, further comprising:
in response to the first response data, displaying a third optotype, wherein:
the third optotype comprises a second set of digital objects having a second size less than the first size in response to the first response data corresponding with the first optotype; and
the third optotype comprises a third set of digital object having a third size greater than the first size in response to the first response data differing from the first optotype by at least a threshold, wherein the visual acuity result is further based on third response data to the third optotype.

20. The method of claim 19, further comprising:
in response to the second response data, displaying a fourth optotype, wherein:
the fourth optotype comprises a fourth set of digital objects having a fourth size less than the first size in response to the second response data corresponding with the second optotype; and
the fourth optotype comprises a fifth set of digital object having a fifth size greater than the first size in response to the second response data differing from the second optotype by at least a threshold, wherein the visual acuity result is further based on fourth response data to the fourth optotype.
